(19) United States
(12) Patent Application Publication
(10) Pub. No.: US 2021/0125732 A1
(43) Pub. Date: Apr. 29, 2021

(54) SYSTEM AND METHOD WITH FEDERATED LEARNING MODEL FOR GEOTEMPORAL DATA ASSOCIATED MEDICAL PREDICTION APPLICATIONS

(71) Applicant: XY.Health Inc., Cambridge, MA (US)

(72) Inventors: Chirag J. PATEL, Boston, MA (US); Arjun K. MANRAI, North Easton, MA (US); Jerod PARRENT, Cambridge, MA (US); Chirag LAKHANI, Cambridge, MA (US)

(73) Assignee: XY.Health Inc., Cambridge, MA (US)

(21) Appl. No.: 17/079,337

(22) Filed: Oct. 23, 2020

Related U.S. Application Data
(60) Provisional application No. 62/926,219, filed on Oct. 25, 2019.

(51) Int. Cl. G16H 50/80; G16H 50/50; G06N 20/00; G06N 5/00

(52) U.S. Cl. CPC G16H 50/80; G06N 5/003; G06N 20/00; G16H 50/50

(57) ABSTRACT

The technology disclosed relates to a system and method for predicting comorbidity trajectories of disease categories on a census tract-basis. The system include logic to process satellite images for a particular census tract and generate respective latent feature vectors for respective satellite images. The system include logic to determine respective weighted average latent feature vectors for the respective latent feature vectors. The respective weighted average latent feature vectors are regressed against a plurality of disease categories and a plurality of risk factors. The regressor generates prevalence scores for disease categories in the plurality of disease categories and for risk factors in the plurality of risk factors. The system can correlate the disease categories with each other and with risk factors to determine comorbidity trajectories of the disease categories in the particular census tract.

### PRIORITY APPLICATION

This application claims the benefit of U.S. Patent Application No. 62/926,219, entitled “SYSTEM AND METHOD WITH FEDERATED LEARNING MODEL FOR GEOTEMPORAL DATA ASSOCIATED MEDICAL PREDICTION APPLICATIONS”, filed Oct. 25, 2019 (Attorney Docket No. XYAI 1000-1). The provisional application is incorporated by reference for all purposes.

### FIELD OF THE TECHNOLOGY DISCLOSED

The technology disclosed relates to use of machine learning techniques to process images and geotemporal data to predict disease prevalence.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves can also correspond to implementations of the claimed technology.

It has been researched and known that human health and life expectancy are influenced by the environment. However, there are very few ways of monitoring health of a population. This monitoring is usually performed in a geographical area. For example, a commonly used geographical unit at which the health of a population is monitored is “county”. Infectious diseases such as COVID-19, SARS, Influenza are commonly reported at a county level. However, county is a large geographical area and effective allocation of resources requires health data of a population at a finer granularity. Disease prevalence, especially for infectious diseases, change over time such as weeks, months, or years, etc. Temporal aspect indicating frequency of change of disease prevalence can be important for public health decision makers when deploying resources to protect their communities.

An opportunity arises to develop a system that can analyze existing available data sources to predict disease prevalence for finer grained geographical areas.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The color drawings also may be available in PAIR via the Supplemental Content tab.

FIG. 1 is a diagram illustrating an exemplary infrastructure to build a data warehouse to be used for federated learning model with multiple edge devices and a central computing cloud, consistent with embodiments of the present disclosure.

FIG. 2 is a diagram illustrating an exemplary system structure to build machine learning algorithm integrating public network and private network, consistent with embodiments of the present disclosure.

FIG. 3 is a flow chart illustrating an exemplary workflow to build and train geotemporal machine learning model, consistent with embodiments of the present disclosure.

FIG. 4 is a diagram illustrating the comparison of public data and the disclosed system predicting the census level disease prevalence and the disclosed system shows superior prediction in four exemplary cities, consistent with embodiments of the present disclosure.

FIG. 5 is a diagram illustrating exemplary area-level satellite image data inputs for automated learning of built environment structures for human disease and behavior prediction or risk profiling, consistent with embodiments of the present disclosure.

FIG. 6 is a diagram illustrating an exemplary system detecting wildfires from space using GOES-16 images, consistent with embodiments of the present disclosure.

FIG. 7 illustrates an architectural level schematic of a system to predict disease prevalence using built environment images, and data from surveys and sensors.

FIG. 8 presents system components of geotemporal data integrator.

FIG. 9 is an example of feature identification from satellite images of built environment.

FIG. 10 is an architectural level schematic of a machine learning model to extract features from satellite images of neighborhoods in cities.

FIG. 11 is an example deep learning pipeline to predict disease prevalence and risk factors per census tract.

FIG. 12 is an example of training the feature extractor using backward propagation and fine-tuning.

FIGS. 13A to 13D illustrate determination of weighted average latent feature vectors for the respective latent feature vectors.

FIG. 14 illustrates an example softmax function.

FIG. 15 presents an example of generating disease and risk factors prevalences using weighted average latent feature vectors as input to respective regressors corresponding to respective disease categories and respective risk factors.

FIG. 16 is a simplified block diagram of a computer system that can be used to implement the technology disclosed.

### DETAILED DESCRIPTION

The following discussion is presented to enable any person skilled in the art to make and use the technology disclosed, and is provided in the context of a particular application and its requirements. Various modifications to the disclosed implementations will be readily apparent to those skilled in the art, and the general principles defined herein may be applied to other implementations and applications without departing from the spirit and scope of the technology disclosed. Thus, the technology disclosed is not intended to be limited to the implementations shown, but is to be accorded the widest scope consistent with the principles and features disclosed herein.

### Introduction

Health of a human population and life expectancy can be influenced by the environmental factors. However, existing monitoring mechanisms and data sources do not provide environmental data at sufficient granularity to support prediction of disease prevalence and risk factors at desired geographical granularity. Prevalence data of many diseases are reported for larger geographical regions such as a “county”. For example, infectious disease prevalence such as COVID-19, SARS, Influenza, etc. are reported at a county level. Other diseases and risk factors such as obesity, diabetes, and cancer are also reported on a county level. A county is a large enough geographical area with many variations geographical place factors such as area-level socioeconomic status, area-level accessibility to resources such as schools, parks, and libraries, etc. Diseases such as mentioned above typically outbreak and occur in a small geographical area than a county. Existing data sources do not provide observations at finer granularity of geographical area.

A second challenge in prediction of disease prevalence is dynamic occurrence of diseases over a period of time. Occurrence of some disease (such as infectious diseases) is more dynamic with high frequency changes within weeks, months, or up to half a year. Some other diseases such as obesity and diabetes can take a longer time to occur in population of a geographic region. The data for high frequency information about health outcomes for units of geographical regions, especially for finer grained locations is difficult to obtain.

The technology disclosed can collect and process information from multiple sources such as satellite image data, data collected from sensors deployed in various geographical areas, and surveys conducted by organizations. The technology disclosed can combine geographical data obtained from satellite images with temporal data collected from sensors, surveys, etc. The technology disclosed can predict disease prevalence at finer grained geographical regions such as neighborhood level areas. Data mobile computing devices that contains medical or health related information can be incorporated using federated machine learning that does not require confidential, proprietary or personal information of users to move outside their computing devices. The technology disclosed therefore includes logic to create geotemporal data by combining geographical and temporal data.

The image data of built environment is associated with census tracts using shapefiles. A two-step deep learning pipeline process satellite image data per census tract to predict disease prevalence and risk factors. In a first step, a pretrained convolutional neural network such as AlextNet (Krizhevsky et al., 2012, “Imagenet classification with deep convolutional neural networks”, published in Advances in Neural Information Processing Systems) or ResNet (He et al. CVPR 2016 available at arxiv.org/abs/1512.03385) is applied to satellite images to extract features of each image per census tract. In one implementation, the features are represented in a 4096-dimensional feature space and are referred to as “latent space features”. In a second step of the deep learning pipeline, the latent space features are processed by a regressor to predict targets in disease prevalence and risk factors from Centers of Disease and Control Protection (CDC) 2017 500 Cities data (available at chronicdata.cdc.gov/browse?category=500+Cities). These outcomes can be organized into health categories and risk factors. Examples of health categories include Arthritis, Current Asthma, High Blood Pressure, Cancer (except skin), High Cholesterol, Kidney disease, COPD, Heart disease, Diabetes, Mental Health, Physical Health, Teeth Loss, Stroke, BP Medication, and Obesity. Examples of risk factors include Health insurance, Annual Checkup, Dental Visit, Cholesterol Screening, Mammography, Pap Smear Test, Colorectal Cancer Screening, Preventive Services (M), Preventive Services (W), Binge Drinking, Smoker, Physical Inactivity, Sleep <7 hours.

The technology disclosed can also predict the disease prevalence and risk factors by using American Community Survey (ACS) Census data provided by United States Census Bureau. In one implementation, the 5 year 2013-2017 ACS Census data which contains sociodemographic prevalence and median values for census tracts is processed by a regressor to predict disease prevalence and risk factors listed above. Examples of sociodemographic variables include the total number of individuals in the tract, proportion of males and females over the age of 65, proportion of individuals by race (e.g., African American White, Hispanic, American Indian, Pacific Islander, Asian, or Other), median income, the proportion of individuals under poverty, unemployed, cohabitate with more than one individual per room, and health insurance status. We present examples of input, machine learning models, and outputs in the following text.

Inputs

The inputs can include satellite imagery data. Examples of satellite image data are presented below.

OpenMapTiles

The images are satellite raster tiles that are downloaded from the OpenMapTiles (available at openmaptiles.com) database (n=4,742,919). The images have a spatial resolution close to 20 meters per pixel allowing a maximum zoom level of 13.1 Images were extracted in tiles from the OpenMapTiles database using the coordinate geometries of the census tracts. After extraction, images were digitally enlarged to achieve a zoom level of 18.

PlanetScope

The PlanetScope images (available at planet.com/products/planet-imagery/) from Planet Labs2 are raster images which have been extracted in a way such that we have complete geometry extractions of the desired census tract. These raster images are extracted in the GeoTIFF format and have a spatial resolution between 3 meters/pixel to 5 meters/pixel which is resampled to provide a 3 meters/pixel resolution thereby allowing a zoom level of somewhere between 13 and 15. Once the geometries are extracted, the images were broken down into tiles for the XYDL pipeline.

SkySat

The SkySat images (available at planet.com) is another product of Planet Labs which has the highest spatial resolution out of all of its products. Similar to PlanetScope images, the SkySat images are complete geometry extractions of the desired census tract. The raster images are extracted in a GeoTIFF format and have a spatial resolution of about 0.72 meters/pixel which is then resampled to 0.5 meters/pixel and thus allowing a zoom level somewhere between 16 and 18. Once the geometries are extracted, the images were broken down into tiles for the XYDL pipeline.

Models

In this section we present details of the example machine learning models that can be trained and applied for prediction during inference.

Current Architecture

First, we passed satellite images through AlexNet, a pretrained convolutional neural network, in an unsupervised deep learning approach called feature extraction. The resulting vector from this process is a “latent space feature” representation of the image comprising 4,096 features. This latent space representation is essentially an encoded (non-human readable) version of the visual patterns found in the satellite images, which, when coupled with machine learning approaches, is used to model the built environment of a given census tract.

For each census tract, we calculated the mean of the latent space feature representation. We performed feature extraction on a NVIDIA Tesla T4 GPU using Python 3.7.7 and the PyTorch package.

Finally, the latent space feature representation is regressed against the disease prevalence and risk features from CDC 500 Cities Project and the demographic factors from the American Community Survey using gradient boosted decision trees. We split the 80% of the data into training and the remaining 20% to testing. To train the model, we used a maximum tree depth of 5, a subsample of 80% of the features per tree, a learning rate (i.e., feature weight shrinkage for each boosting step) of 0.1, and used 3-fold cross-validation to determine the optimal number of boosted trees. Training was completed on a NVIDIA Tesla T4 GPU using Python 3.7.7 and the XGBoost package.

In a separate analysis, both satellite image features and the social determinants of health features from the American Community Survey were regressed against the CDC 500 Cities Project in the same manner.

Interpretation of the Model

The inputs of our model are satellite image latent feature vectors. These vectors represent the elements of the environment that are detectable from the satellite images. Such features include buildings, roads, highways, trees, parks, sidewalks, walking paths, and farmland. These features are indicative of the exposures in a community that contribute to the community's health and disease risk. For example, a community with a higher density of buildings and highways would have a higher health risk for asthma. Similarly, a community with many walking paths would have greater access to physical activity, and lower risk for heart disease.

We have prototyped pipelines to determine what features of the environment are being focused on by these black box models. In the image below, we can see that the model is identifying a number of large, dense buildings in a city block, and correlating these features to the health indicator.

Architectures

This architecture is similar to the current architecture, however there will be some updates to the pipeline after extracting the image features. We are currently prototyping the following.

Multilayer Perceptron

This approach begins similarly to the approach described above. Features are extracted from AlexNet and the resulting 4,096 feature vector is averaged across images within the same census tract.

However, the regression differs: We perform the regression of the latent space feature representation against the disease prevalence and risk features from CDC 500 Cities Project using a Multilayer Perceptron (MLP). The MLP is sequential with three hidden layers. The first hidden layer has 1,024 nodes, the second has 512 nodes, and the third has 512 nodes. All layers have a ReLU activation function and a dropout layer with 10% probability. We split the data into 60% training, 20% validation, and 20% testing. The model is trained for the optimal number of epochs as determined by the validation set. We use the Adam optimizer and a learning rate of 0.0001 with 0.01 weight decay.

Backpropagation of Errors Through the Mean and Fine-Tuning of Pretrained AlexNet

This approach allows for fine-tuning of the mean latent space feature vector that is used in regression. The regression is performed using an MLP as described in the Multilayer Perceptron section above. However, when training the MLP, the loss from prediction is backpropagated through the mean function and used to fine-tune (i.e., adjust the weights slightly) the AlexNet feature extractor. As a result, the latent space feature vector is no longer just the pretrained representation but rather is calculated as a function of the outcome variable.

The MLP is sequential with two hidden layers. The first hidden layer has 512 nodes and the second has 256 nodes. All layers have a ReLU activation function and a dropout layer with 10% probability. We split the data into 60% training, 20% validation, and 20% testing. We use the Adam optimizer and a learning rate of 0.001 with 0.01 weight decay. We unfreeze the last convolutional layer from the pretrained AlexNet base to fine-tune.

Utilization of ResNet Architecture

This approach is similar to the Current Approach described above; however, instead of using a pretrained AlexNet as the convolutional base, we use a pretrained ResNet-151 or ResNet-50. We then use the latent space feature representation from the ResNet to perform gradient boosted trees regression in the case of the ResNet-151, and MLP regression in the case of the ResNet50, as described above.

Weighted Average Across Feature Vectors Rather Than Simple Average

This approach does not fine-tune the feature extractor. Instead, this approach attempts to understand the importance of all the features in the latent space feature vector for each image and use this newfound knowledge in a learned weighting scheme rather than simply taking the mean over all the extracted feature vectors.

Feature vectors are extracted from a pretrained AlexNet and are of size 4096 dimensions. The architecture is the same as that of the Multilayer Perceptron, but additionally contains a 4096-dimension learned parameter vector that is dot product-ed with each image feature vector to produce an unnormalized image weight. The unnormalized image weights are then passed through a softmax function to get a normalized image weight over all images in a census tract. These normalized weights are used to take a weighted average of all the image feature vectors in a census tract.

Outputs

In this section we present details of the example outputs from the machine learning models presented above.

American Community Survey

This data is 5-year 2013-2017 American Community Survey (ACS) Census data6, which contains sociodemographic prevalences and median values for census tracts. These data contain demographic variables, including the total number of individuals in the tract, proportion of males and females over the age of 65, proportion of individuals by race (e.g., African American, White, Hispanic, American Indian, Pacific Islander, Asian, or Other), median income, the proportion of individuals under poverty, unemployed, cohabitate with more than one individual per room, and health insurance status.

Outcomes: CDC 500 Cities Project

The disease prevalence and risk factors data is sourced from the US Centers for Disease Control and Prevention 2017 500 Cities data.7 The 500 Cities data contains disease and health indicator prevalence for 26,968 individual census tracts of the 500 Cities which are the most populous in the United States. These prevalences are estimated from the Behavioral Risk Factor Surveillance System.

The disease prevalence and risk factors is used as the outcome data for the XYDL pipeline and includes the following fields.

Health Categories: Arthritis, Current Asthma, High Blood Pressure, Cancer (except skin), High Cholesterol, Kidney Disease, COPD, Heart Disease, Diabetes, Mental Health, Physical Health, Teeth Loss, Stroke, BP Medication, Obesity.

Risk Factors: Health Insurance, Annual Checkup, Dental Visit, Cholesterol Screening, Mammography, Pap Smear Test, Colorectal Cancer Screening, Preventive services (M), Preventive services (W), Binge Drinking, Smoker, Physical Inactivity, Sleep <7 hours.

We have predicted all of the above targets as prevalence (ranging from 0-1).

Other outcomes of interest include additive (sum of prevalence for a outcome) to assess multi-morbidity. Please see COVID-19 disclosure for assessing multimorbidity via unsupervised approach.

### Overview

Many alternative embodiments of the present aspects may be appropriate and are contemplated, including as described in these detailed embodiments, though also including alternatives that may not be expressly shown or described herein but as obvious variants or obviously contemplated according to one of ordinary skill based on reviewing the totality of this disclosure in combination with other available information. For example, it is contemplated that features shown and described with respect to one or more embodiments may also be included in combination with another embodiment even though not expressly shown and described in that specific combination.

For purpose of efficiency, reference numbers may be repeated between figures where they are intended to represent similar features between otherwise varied embodiments, though those features may also incorporate certain differences between embodiments if and to the extent specified as such or otherwise apparent to one of ordinary skill, such as differences clearly shown between them in the respective figures.

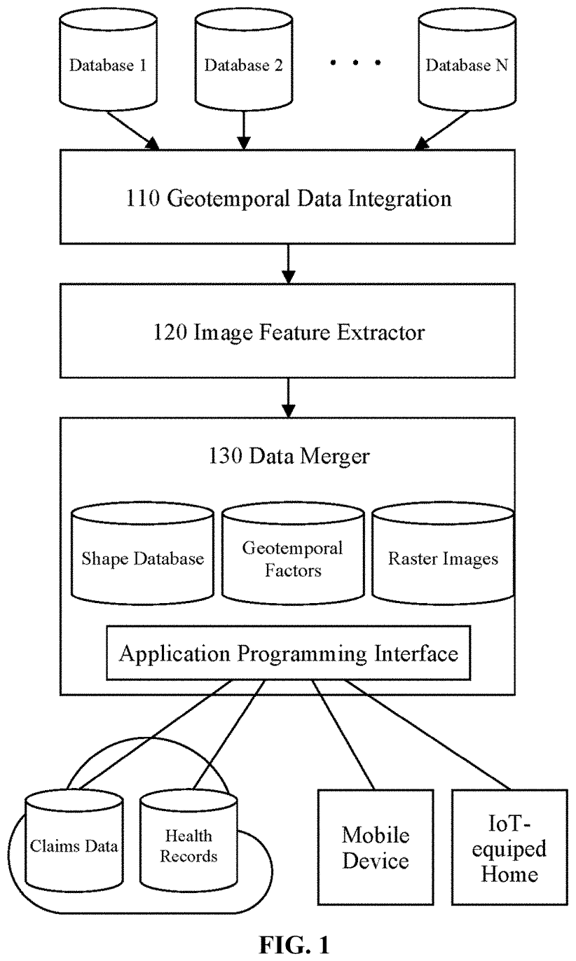

Reference is now made to FIG. 1, which is a diagram illustrating an exemplary infrastructure to build exposome data warehouse to be used for federated learning model with multiple edge devices and a central computing cloud, consistent with embodiments of the present disclosure.

Extracting geotemporal factors put certain requirements on the data collected to be qualified for the use in a machine learning module configured to personalize medical prediction in association with geotemporal factors. Normal geographical data collected by institutes, statisticians, and public databases are required to be labelled together with time. Such geographical data is characterized as having a geographical identifier, which is an identifiable point in X-Y coordinate system. Examples of data with geographical identifier include latitude and longitude information, region or area in a census tract, postal/zip code of a country, or a geographical shape. A time identifier, which is to be associated with a geographical identifier, may be time information with second, minute, hour, day, month, and year. Data associated with the geographical identifier and time identifier can be, but not limited to, air pollution level data of a region with geographical coordinate point and hourly frequency of updating, median income of a region with yearly frequency of updating, a raster-based image of a region with date, etc.

Information in practice which is up to the level for machine learning module configured to personalize medical prediction in association with geotemporal factors can be obtained from different sources. In some embodiments of the module, data with geotemporal factors are retrieved from public database of government authorities. National Oceanic Administration Association provides weather data with geographical and time identifiers. Environmental Protection Agency provides air pollution data with geographical and time identifiers. United States Census provides regional socioeconomic data with time identifier. In some embodiments of the module, data with geotemporal factors are retrieved from non-public database. For instances, point sensors can be used to detect, collect, and obtain noise or radiation of a region at a point of time, satellite images can be used to obtain rasterized images of the earth planet.

Geotemporal data, along with its geographical identifier and temporal identifier, obtained from aforementioned sources, are to be unified in a geotemporal data integrator 110. Geotemporal data integrator 110 is configured to integrate geotemporal data obtained from various information sources. In some embodiments, the geotemporal data integrator is configured to utilize spatial and object-relational database management technologies provided by open source mapping servers, such as Postgress geographical information system data base technology.

Aggregated and integrated geotemporal data out of geotemporal data integrator are to be sent to an image feature extractor 120. Image feature extractor 120 is configured to derive non-redundant and informative values, i.e., features, of the aggregated and integrated geotemporal data to facilitate the subsequent learning and generalization steps. In some embodiments, image feature extractor 120 is configured to reduce dimensions of vectors, leading to better human interpretations. The initial set of integrated geotemporal data is reduced to more manageable features or factors for processing, but still accurately and completely describing the original integrated geotemporal data set. Depending on the purpose of the data analysis and feature of the geotemporal data, the algorithm can be designed to count smokestacks in an image with buildings, or to count the number of automobiles on a high-way image, etc. In some embodiments, image feature extractor 120 is configured to extract features include without limitation fires, air pollution, and census tract information (i.e., regional income). When in rural areas where there is no census data available, image feature extractor 120 is configured to predict census-level information, e.g., regional income, as a function of image data.

In addition, image feature extractor 120 is also configured to replace missing data with substituted values. Regional information can be derived from a multiple point estimates in the process of imputation. For instance, from a triangle of air pollution sensors, region-level aggregate air pollution can be inferred.

Thereafter, geotemporal data processed in the image feature extractor is to be sent to a data merger 130. Data merger 130 can be viewed as a giant database, specifically, also known as exposome data warehouse, with an application programming interface. An integrated data store or an integrated data warehouse is configured to comprise one or more database. One database or one of the databases is configured to store the processed geotemporal data output from image feature extractor 130.

In some embodiments, exposome data warehouse comprises a shape database. The shape database stores geometry information which defines borders or contours of locations. For example, a group of geometry information can be adopted to represent a shape of a governmental administrative region, e.g., a city, a county, a state, etc., that constitute the border lines of the region. A physical location can be represented and queried by numbers, strings, etc., stored in the shape database. The group of geometry information, or shape data, in combination of geographical identifier and time identifier, can be used to represent geotemporal situation of a region at certain time period of interest through access via Application Program Interface (or API).

In some embodiments, exposome data warehouse comprises a raster image database. Raster image, also known as raster graphics or bitmap image, is a dot matrix data structure that represents a generally rectangular grid of pixels viewable via a monitor, paper, or other display medium. Each pixel is represented by a point of color in red, green, and blue (RGB). Large amount of raw image data of earth surface constitutes the raster image database. Similarly, raster image data are to be processed to extract features of a region by machine learning algorithms and then to be integrated into extracted features from other data sources and draw health patterns.

Data merger 130 is also configured to comprise an Application Programming Interface. The Application Programming Interface is an access point to allow compatible application programs to access geotemporal data stored in data merger 130. The Application Programming Interface is configured to extract information from data merger 130 and pack such extracted information to be used by downstream computational processes. The downstream computational processes are stored and pre-installed in other part of the information infrastructure, most of the time physically separated and apart from the Exposome data warehouse, but electrically coupled through internet or any other communication network.

In some embodiments, such downstream computational process exists in another pre-aggregated individual data assembly where plurality of individual personal healthcare related information is assembled and aggregated. This individual data assembly is populated data comprising multiple, usually large amount of healthcare information of population. Examples of such data assembly include medical claim data available to medical insurance issuers, or healthcare records of patients available to healthcare provider. The individual data assembly also comprises a Cohort that emerges from the populated data.

In some other embodiments, such downstream computational process exists in another edge device where individual data including healthcare related or non-healthcare related information is stored. Plurality of such individual data from plurality edge devices can be configured to connected to the exposome data warehouse via application programming interface. Examples of such edge device included personal mobile device which stores personal data, or Internet-of-Things sensor which stores data of a house, a car, or any equipment the sensor is installed to, e.g., household device. Data stored in these edge devices is device-specific data. Out of consideration to protect privacy and information security, certain data stored in these edge devices may never leave the edge devices and are processed in the edge devices.

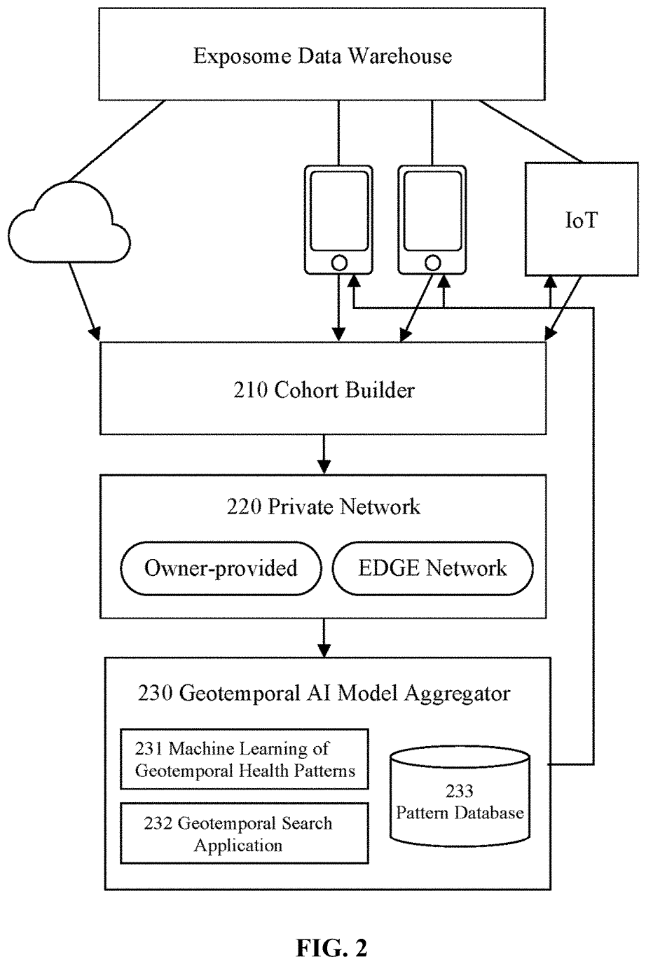

Reference is now made to FIG. 2, which is a diagram illustrating an exemplary system structure to build machine learning algorithm integrating public network and private network, consistent with embodiments of the present disclosure. The system to build machine learning algorithm integrating public network and private network comprises a cohort builder 210, a distributed learning network 220, and a geotemporal AI model aggregator 230.

Cohort builder 210 is configured to receive data from the pool of pre-aggregated individual data assembly, edge device data, and exposome data warehouse data, to assemble cohorts of certain characteristics of data. Such assembled data by cohort builder 210 shares certain common characteristics retrieved by setting common inclusion or exclusion criteria from the data of the pool. As an example, inclusion or exclusion criteria can be individual with a specific disease versus healthy controls. As another example, inclusion or exclusion criteria can be a machine learning model type, such as regression model, neural nets model, tree-based model, etc. As another example, inclusion or exclusion criteria can be a set of model parameters. As another example, inclusion or exclusion criteria can be a random or a pseudo random allocation of training and independent training dataset.

In some embodiments, cohort builder 210 is achieved by building a digital twin. A digital object or system (that represents a human) is built by mimicking the biological characteristics of a real-world physical object or system. The digital object or system is to develop a mathematical model that simulates the real-world original in digital space. The digital twin is constructed to receive inputs from data from real-world counterpart. Therefore, the digital twin is configured to simulate and offer insights into performance and potential problems of the human counterpart.

In contrast to cohort building 210 to assemble data with common characteristics and build digital system for simulation based on assembled data, distributed learning network 220 is configured to perform supervised learning to model human disease or other health related characteristics as a function of geotemporal factors in a distributed learning or federated learning manner, utilizing data from a plurality of edge devices. Federated learning cloud is partly replaced by the crowd of end users who use application programs by which edge devices collect data, train, compute, and evaluate data stored in devices these application programs run on. Edge devices federate data by sending derived insights, which are bunches of tensors technically, to a computing cloud. The bunch of tensors as derived insights are then to be averaged in the computing cloud.

In some embodiments, the computing cloud can be an owner-provided private network, which is used to assemble derived insights from various edge devices of the owner. In some embodiments, the computing cloud can be an edge network, which is a public cloud network and used to assemble derived insights from various edge devices of a plurality of end users. In some embodiments, the computing cloud can be an edge network, which is a private network deployed by a private company and used to assemble derived insights from various edge devices of a plurality of end users of the private company, to protect information security and privacy of these end users, usually the private company's clients. In the embodiments with privately owned network, federated learning algorithms are configured to operate within firewall of owners' edge devices, e.g., smart phone, or firewall of database, i.e., relational database residing within firewall of a research institute or company.

Again, standard machine learning approaches require all learning and training data to reside in a centralized cloud database. Optimization procedure optimizes a model as a function of predictor variables, e.g., geotemporal variables, that best predicts the known outcome or dependent variable, e.g., health indicators such as disease or phenotype like age or body mass index. In the case of federated learning, the learning method is delivered to where the data resides, i.e., edge devices or database, from a public cloud provider. The learning method sends the contribution of the optimization procedure, and not the individual private data, for that one data point or database back to the public cloud provider to update the machine-learned algorithm. No individual-level data is stored outside of edge devices or firewall of private network.

Geotemporal AI model aggregator 230 is configured to receive averaged derived insights to update machine learning models. Geotemporal AI model aggregator 230 comprises machine learning model of geotemporal health pattern 231, one or more geotemporal search application 232, and a pattern database 233.

Geotemporal AI model aggregator 230 can be configured to work in a public computing cloud or a private computing cloud. Within which, machine learning model of geotemporal health patterns 231 is configured to received averaged derived insights, i.e. learned average value of bunch of tensors, from distributed learning network 220. Machine learning model of geotemporal health pattern 231 is to be updated by the averaged derived insights and therefore further improve the learning model. Improved learning model is to be sent to edge devices for improved federated learning. Geotemporal search application 232, an application program, is configured to search geotemporal data. Pattern database 233 is configured to stores data of various patterns which are geographical and may have impact on health condition of human being, direct or attenuated. Machine learning model of geotemporal health pattern 231 is initially build with the facilitations of geotemporal search application 232 and pattern database 233.

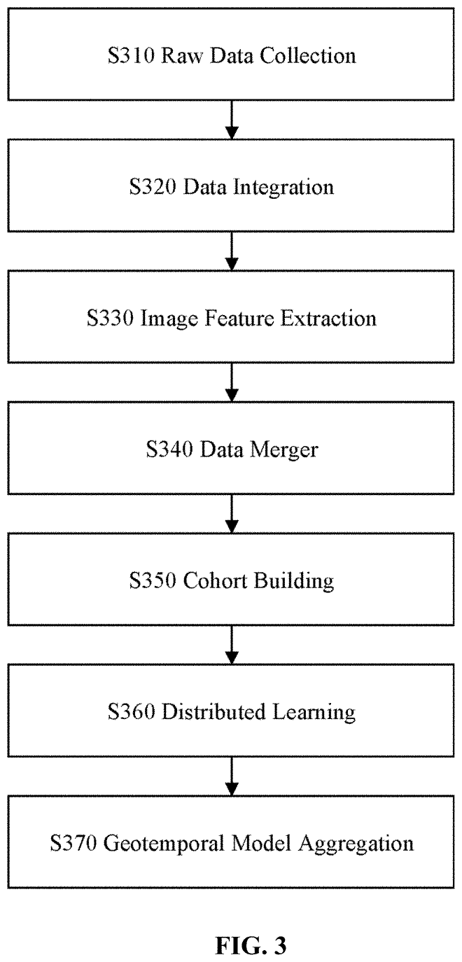

Reference is now made to FIG. 3, which is a flow chart illustrating an exemplary workflow to build and train geotemporal machine learning model, consistent with embodiments of the present disclosure.

The workflow to build and train geotemporal machine learning model comprises step S310 raw data collection, step S320 data integration, step S330 image feature extraction, step S340 data merger, step S350 cohort building, step S360 distributed learning, and step S370 geotemporal model aggregation.

In step S310, qualified raw data are to be collected. To be qualified as raw data used for machine learning model building purpose, normal geographical data need to be associated with a time identifier. When a geographical identifier and a time identifier combined, the information is turned into geotemporal data, which is ready for geotemporal factor extraction at later stage of the method. A plurality of public database having raw data with geotemporal factors are available to retrieve data from, such as weather data with location and time, air pollution data with location and time, socioeconomic data of a region with time, etc. Furthermore, a plurality of non-public database having raw data with geotemporal factors are available to retrieve data from, such as noise or radiation data or a region at a point of time, rasterized images of regions of the earth from satellite with time, etc. Qualified raw data are collected and gathered together in step S310.

In step S320, collected qualified raw data are integrated to suit model building requirement at next steps. As an example, spatial and object-relational database management technologies provided by open source mapping databases can be utilized to integrate qualified raw data from a plurality of source database into geotemporal data with geographical factors and proper time label.

In step S330, features of images are to be extracted. The features are non-redundant and informative values, while representing information sufficient to facilitate subsequent learning and generalization requirements. Dimensions of vectors can be reduced for better human interpretations. The initial set of integrated geotemporal data is reduced to more manageable features or factors for processing, but still accurately and completely describing the original integrated geotemporal data set. Depending on the purpose of the data analysis and feature of the geotemporal data, step S330 can be designed to count smokestacks in an image with buildings, or to count the number of automobiles on a highway image, etc. Meanwhile, missing data can be replaced by substituted values in step S330. By imputation, information about a region can be derived from a plurality of point estimates. Step S330 is an image-to-feature creation step by imputation in computation. It adapts to derive a plurality of machine-learned annotation of factors.

In step S340, various data, including the machine-learned annotation of factor data, are merged in a giant database. For example, in one implementation the database can be an exposome data warehouse. These machine-learned annotations of factors, also called geotemporal factors are linked through various shape data, as one unique geometric shape representing one specific region or location of the world. On the other side, these geotemporal factors are also linked through in association with raster images. Similarly, raster image data are to be processed to extract features of a region by machine learning algorithms and then to be integrated into extracted features from other data sources and draw health patterns.

Also, in step S340, exposome data warehouse is adapted to interact with external devices or network via an application programming interface. It allows compatible application programs to access geotemporal data stored in exposome data warehouse. Through the application programming interface, downstream computational process is enabled to process merged data.

In step S350, cohorts are built based on exposome data warehouse data, along with pre-aggregated individual data assembly, individual edge device data, or household edge device data, etc. Cohorts of certain characteristics of data can be built in this step. Data sharing common characteristics are to be retrieved by setting common inclusion or exclusion criteria from the data of the pool. As an example, inclusion or exclusion criteria can be individual with a specific disease versus healthy controls. As another example, inclusion or exclusion criteria can be a machine learning model type, such as regression model, neural nets model, tree-based model, etc. As another example, inclusion or exclusion criteria can be a set of model parameters. As another example, inclusion or exclusion criteria can be a random or a pseudo random allocation of training and independent training dataset.

In step S360, supervised distributed or federated learning to model human disease or other health related characteristics as a function of geotemporal factors are executed. Data can be from a plurality of edge devices. The plurality of edge devices each has application program running on to evaluate data stored in the devices. Derived insights, which are bunches of tensors, are to be sent to a computing cloud, where these bunches of tensors are averaged. Highly sensitive personal and private data are retained in edge devices by this way, privacy concerns are much eased accordingly.

In step S370, a geotemporal machine learning model is aggregated, specifically, a machine learning model of geotemporal health pattern is to be updated by averaged derived insights and be further improved. Pattern database storing data of various patterns which are geographical and may have impact on health condition of human being, direct or attenuated, is utilized to improve the geotemporal machine learning model also.

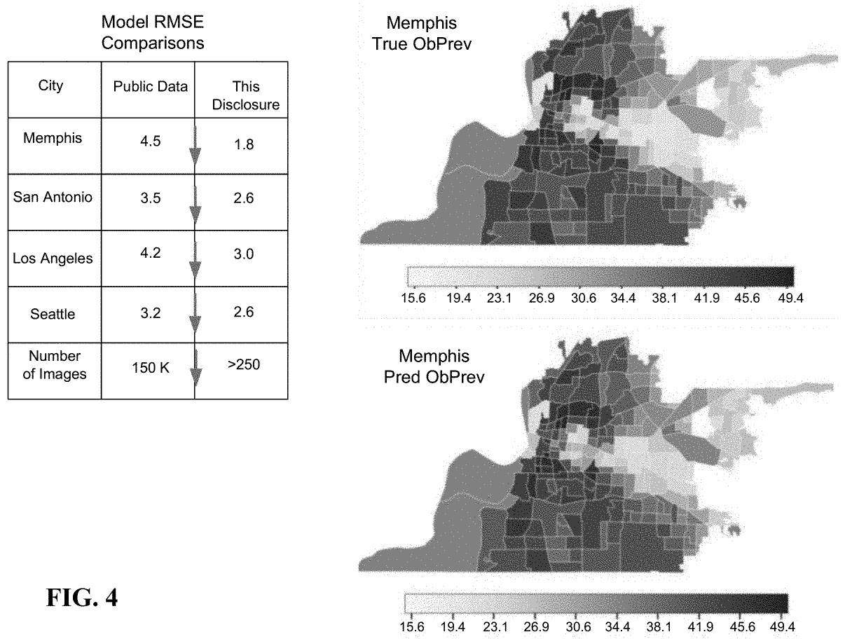

Reference is now made to FIG. 4, which is a diagram illustrating the comparison of public data and the disclosed system predicting the census level disease prevalence and the disclosed system shows superior prediction in four exemplary cities, consistent with embodiments of the present disclosure.

In some embodiments, the system is configured to predict prevalence of obesity, diabetes, heart disease, and other health indicators. Specifically, the deep learning algorithm is configured to transfer a model trained on a corpus of internet images and then be retrained on satellite map images (e.g., OpenStreetMap or Google). Features of the built environment predicted up to 65% of variation in obesity prevalence and the root mean square error for four exemplary cities, Memphis, San Antonio, Los Angeles, and Seattle is 1.8, 2.6, 3, and 2.6, respectively. The deep learning system is configured to input large number of images, e.g., 250,000 image data of each census tract and integrated the two across space and predicted the prevalence of the census-tract disease prevalence.

In some embodiment, the disclosed method can be configured to predict comorbidities and or trajectories to disease when phenotypes arise from others, or, are “comorbid” with other phenotypes. For instance, obesity and type 2 diabetes. Or, some phenotypes can be thought of as trajectories. For instance, obesity to type 2 diabetes, further to heart disease; or, obesity to type 2 diabetes, further to kidney disease. In these scenarios, if the disclosed method is configured to predict obesity as a function of exposome and geotemporal factors, type 2 diabetes, and further heart disease or kidney disease can also be predicted by shared geotemporal factors or correlated risk factors. The probability of type 2 diabetes, and heart disease or kidney disease as a function of geosurveillance features can be tested.

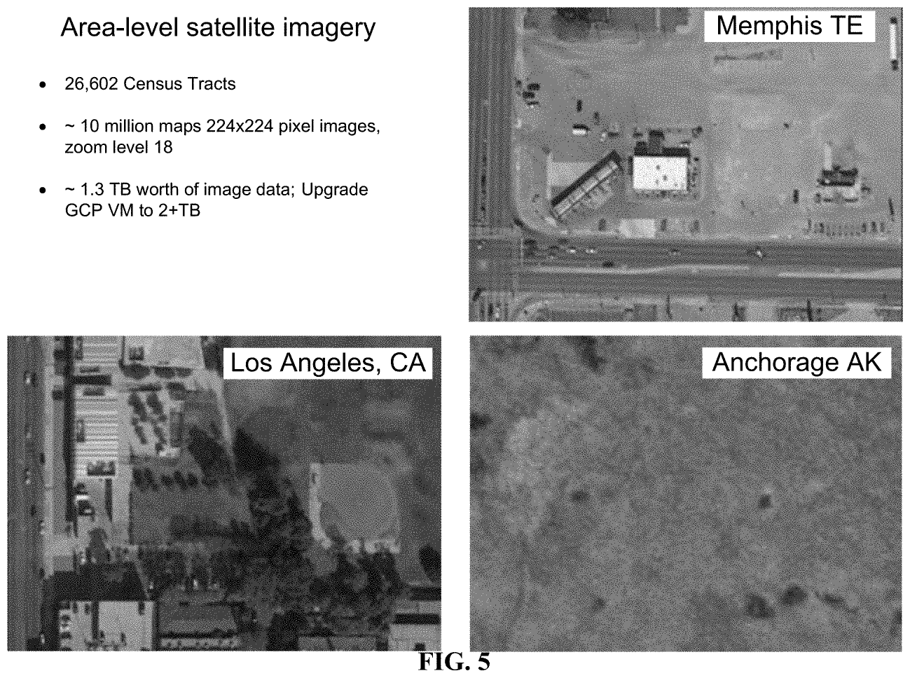

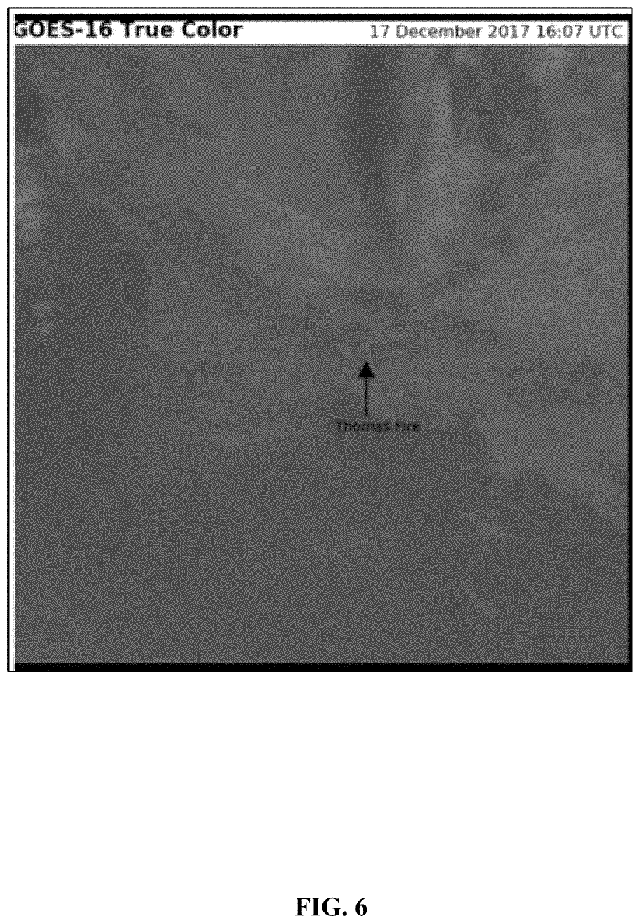

Reference is now made to FIG. 5, which is a diagram illustrating exemplary area-level satellite image data inputs for automated learning of built environment structures for human disease and behavior prediction or risk profiling, consistent with embodiments of the present disclosure.

In some embodiments, individuals or population at risk can provide their coordinates, an address or a list of addresses, which can be mapped to location(s) on the earth. The system is configured to query area-level image information from the database and leverage machine learning algorithms, to provide a risk profile for individuals or population.

It is to be understood that, because some of the constituent system components and method steps depicted in the accompanying figures can be implemented in Software, the actual connections between the systems components (or the process steps) may differ depending on the fashion in which the present disclosure is programmed. Given the teachings of the present disclosure provided herein, one of ordinary skill in the similar art will be able to contemplate these and similar implementations or configurations of the present disclosure.

It is to be understood that the configuration and boundaries of the functional building blocks of system have been defined herein for the convenience of the description. Alternative boundaries may be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. While the present disclosure has been described in detail with reference to exemplary embodiments, those skilled in the similar art will appreciate that various modifications and substitutions can be made thereto without departing from the spirit and scope of the disclosure.

Method and system with federated learning model for healthcare application in association with geotemporal factor are disclosed. The system comprises a raw data collector to collect geographical data associated with time identifier from database, a data integrator to integrate geographical data with corresponding time identifier, an image feature extractor to extract geotemporal information and reduce dimensions of vectors, a data merger to merge geotemporal information with geometric shape information, a cohort builder to build cohort of data based on criteria of data inclusion from pool of data of data merger, cloud, or edge device, a distributed learning network to learn from tensors sent by edge or cloud device, and a geotemporal machine learning model aggregator to receive averaged value of tensors to update the geotemporal machine learning model.

### Environment

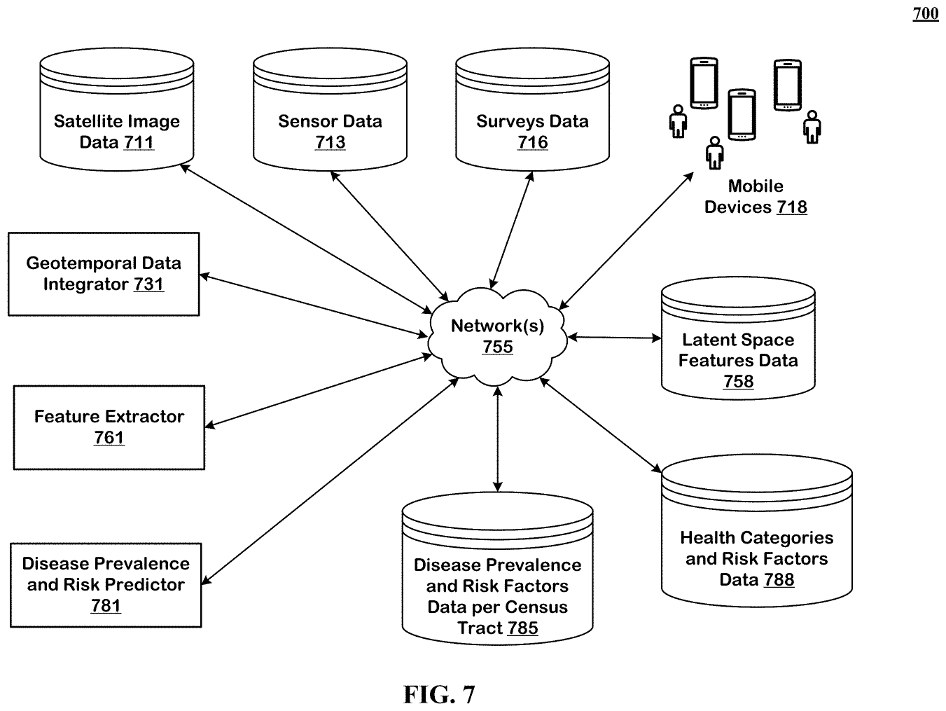

We describe a system for predicting census tract-level disease prevalence and risk factors using satellite images of built environment. The system is described with reference to FIG. 7 showing an architectural level schematic of a system in accordance with an implementation. Because FIG. 7 is an architectural diagram, certain details are intentionally omitted to improve the clarity of the description. The discussion of FIG. 7 is organized as follows. First, the elements of the figure are described, followed by their interconnection. Then, the use of the elements in the system is described in greater detail.

FIG. 7 includes the system 700. This paragraph names labeled parts of system 700. The system includes a Geotemporal data integrator 731, a feature extractor 761, a disease prevalence and risk score predictor 781, a satellite image database 711, a sensor data database 716, mobile devices 718, a latent space features database 758, a health categories and risk factors database 788, a disease prevalence and risk factors database per census tract 785, and a network(s) 755.

The technology disclosed can use satellite image data of built environment for census tract-level communities for predicting disease prevalence and risk factors sourced from US Centers for Disease Control and Prevention 2017 500 cities data. The technology disclosed can include satellite image data from various sources to predict disease prevalence and risk factors. Examples of satellite images data sources include OpenMapTiles (available at openmaptiles.com), PlanetScope (available at planet.com/products/planet-imagery/), SkySat (available at planet.com), etc. The images can be stored in the satellite image database 711.

The OpenMapTiles are satellite raster tiles downloaded from the OpenMapTiles database (n=4,742,919). The images have a spatial resolution close to 20 meters per pixel allowing a maximum zoom level of 13. Images are extracted in tiles from the OpenMapTiles database using the coordinate geometries of the census tracts. After extraction, images were digitally enlarged to achieve a zoom level of 18. The images can be stored in the satellite image database 711.

The PlanetScope images from Planet Labs are raster images which have been extracted in a way such that we have complete geometry extractions of the desired census tract. These raster images are extracted in the GeoTIFF format and have a spatial resolution between 3 meters/pixel to 5 meters/pixel. The images are resampled to provide a 3 meters/pixel resolution thereby allowing a zoom level between 13 and 15. Once the geometries are extracted, the images are broken down into tiles for the XYDL pipeline. The images can be stored in the satellite image database 711.

The SkySat images is another product of Planet Labs which has the highest spatial resolution out of all of its products. Similar to PlanetScope images, the SkySat images are complete geometry extractions of the desired census tract. The raster images are extracted in a GeoTIFF format and have a spatial resolution of about 0.72 meters/pixel which is then resampled to 0.5 meters/pixel and thus allowing a zoom level somewhere between 16 and 18. Once the geometries are extracted, the images were broken down into tiles for the deep learning pipeline. The images can be stored in the satellite image database 711.

The technology disclosed can also collect and use sensor data for use in prediction of disease prevalence and risk factors. Data collected from sensors deployed in various geographical areas deployed by individuals, organizations or government. For instance, from a triangle of air pollution sensors, region-level aggregate air pollution can be inferred. Point sensors can be used to detect, collect, and obtain noise or radiation of a region at a point of time. Data can be collected from Internet-of-Things (IoT) sensors which store data of a house, a car, or any equipment the sensor is installed to, e.g., household device. The data from sensors can be stored in the sensor database 713 and merged with satellite image data on a census tract level to provide additional input for prediction of disease prevalence and risk factors.

The technology disclosed can use American Community Survey (ACS) Census data provided by United State Census Bureau to predict disease prevalence and risk factors per census tract. The data is a 5-year 2013-2017 American Community Survey (ACS) Census data, which contains sociodemographic prevalences and median values for census tracts. These data contain demographic variables, including the total number of individuals in the tract, proportion of males and females over the age of 65, proportion of individuals by race (e.g., African American, White, Hispanic, American Indian, Pacific Islander, Asian, or Other), median income, the proportion of individuals under poverty, unemployed, cohabitate with more than one individual per room, and health insurance status. The ACS data can be saved in surveys database 716.

The system can collect and store data from edge devices including mobile devices 718 which can store personal health records, insurance, prescription records, etc. The data from edge devices need not travel to a central database for security and privacy reasons. The system can include logic to build exposome data warehouse to be used for federated learning model with multiple edge devices and a central computing cloud. The technology disclosed can combine geographical data from satellite images with temporal data from sensors, surveys and edge devices to create geotemporal data using the geotemporal data integrator 731.

The system can use transfer learning using pretrained machine learning models. Transfer learning can include fine-tuning a pretrained machine learning model for a new task or using the pretrained machine learning model a feature extractor. The system can use a pretrained AlexNet, a convolutional neural network (CNN) or a pretrained ResNet, a residual convolutional neural network as feature extractor in the deep learning pipeline. In one implementation, the satellite images are passed through the pretrained AlexNet producing “latent space features” that are vectors in a 4096-dimensional space. The latent space representation of satellite images is an encoded (non-human readable) version of the visual patterns found in the satellite images. The features in the 4096-dimensional space can be used to model the built environment of a given census tract. The latent space features can be stored in the latent space features database 758.

The latent space features per census tract are passed through a regression model in a second step of the deep learning pipeline to predict outcomes for disease prevalence and risk factors from CDC 500 Cities Data. Examples of regression models include Extreme Gradient Boosting (or XGBoost) model or multilayer perceptron-based regression model. The disease prevalence and risk predictor 781 includes logic to process the latent space features by applying a regression model and predict outcomes for the disease prevalence and risk factors. The system can predict specific health categories and risk factors in a range from 0 to 1. The health categories and risk factors can be stored in the health categories and risk factors database 788.

Completing the description of FIG. 7, the components of the system 700, described above, are all coupled in communication with the network(s) 755. The actual communication path can be point-to-point over public and/or private networks. The communications can occur over a variety of networks, e.g., private networks, VPN, MPLS circuit, or Internet, and can use appropriate application programming interfaces (APIs) and data interchange formats, e.g., Representational State Transfer (REST), JavaScript Object Notation (JSON), Extensible Markup Language (XML), Simple Object Access Protocol (SOAP), Java Message Service (JMS), and/or Java Platform Module System. All of the communications can be encrypted. The communication is generally over a network such as the LAN (local area network), WAN (wide area network), telephone network (Public Switched Telephone Network (PSTN), Session Initiation Protocol (SIP), wireless network, point-to-point network, star network, token ring network, hub network, Internet, inclusive of the mobile Internet, via protocols such as EDGE, 3G, 4G LTE, Wi-Fi and WiMAX. The engines or system components of FIG. 7 are implemented by software running on varying types of computing devices. Example devices are a workstation, a server, a computing cluster, a blade server, and a server farm. Additionally, a variety of authorization and authentication techniques, such as username/password, Open Authorization (OAuth), Kerberos, Secured, digital certificates and more, can be used to secure the communications.

### System Components

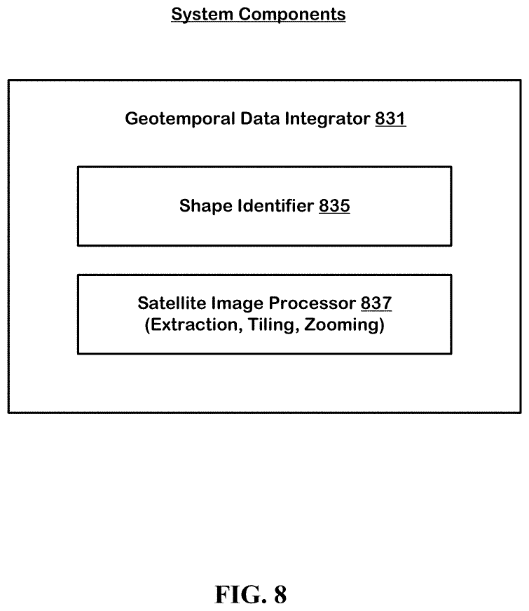

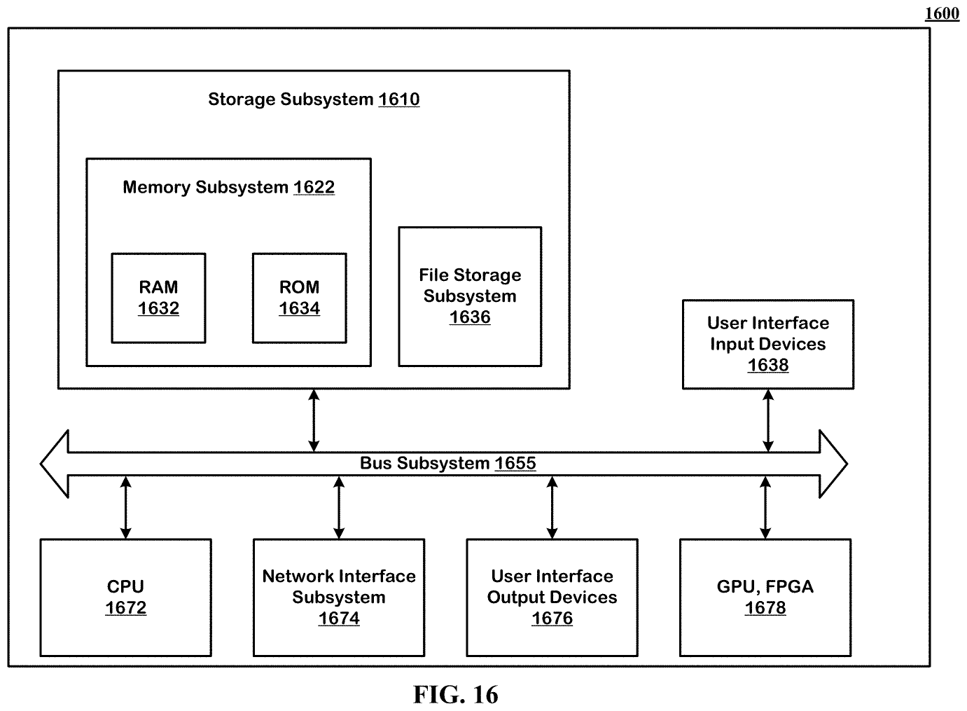

FIG. 8 is a high-level block diagram of components of geotemporal data integrator 831. These components are computer implemented using a variety of different computer systems as presented below in description of FIG. 16. The illustrated components can be merged or further separated, when implemented. Geotemporal data integrator 831 comprises of a shape identifier 835, and a satellite image processor 837.

Geographical data is characterized as having geographical identifier which is an identifiable point in X-Y coordinate system. Examples of data with geographical identifier include latitude and longitude information, region or area in a census tract, postal or ZIP codes, or a geographic shape. The system can include a shape database. The shape identifier 835 includes logic to use the information stored in the shape database to borders and contours of locations. The shape database stores geometry information which defines border and contours of locations. For example, a group of geometry information can be adopted to represent a shape of a governmental administrative region, e.g., a city, a county, a state, etc., that constitute the border lines of the region. A physical location can be represented and queried by numbers, strings, etc., stored in the shape database. The group of geometry information, or shape data, in combination of geographical identifier and time identifier, can be used to represent geotemporal situation of a region at certain time period of interest through access via Application Program Interface.

Satellite image processor 837 can include logic to extract the satellite images from various data sources using the coordinate geometry of census tracts. The images are broken down into tiles for processing by the deep learning pipelines. In one implementation, the image feature extractor can take images in 224 pixel by 224 pixel sizes. The images from different data sources can be processed to achieve a desired zoom level for further processing.

The geotemporal data integrator can combine geographic and temporal data from different sources to create a geotemporal data. A time identifier, which is to be associated with a geographical identifier, may be time information with second, minute, hour, day, month, and year. Data associated with the geographical identifier and time identifier can be, but not limited to, air pollution level data of a region with geographical coordinate point and hourly frequency of updating, median income of a region with yearly frequency of updating, a raster-based image of a region with date, etc. The technology disclosed can process geotemporal data to predict comorbidity trajectories of disease categories and risk factors on a census tract-basis.

The geotemporal data integrator can include logic to combine non-image-based data with satellite image data. In one implementation, the non-image-based data are merged with image features extracted from the feature extractor. For example, suppose we have “Y” variable indicating disease prevalence, measured on a census tract unit. Suppose we have an “X” variable, such as median income OR percent Mexican, also on a census tract unit. These data, for example, can come from American Community Survey (Census). assume that we have multiple of satellite images that are images of a census tract. We feed each satellite image through a deep neural network and output a latent space feature vector in a 4096-dimensional image space. For each census tract, we take the mean of all the 4,096 feature vectors for that census tract. This results in a 4,096-dimensional feature vector that represents one census tract. The geotemporal data integrator can include logic to do a column-wise append of the X variables to the 4,096-dimensional feature vector. The resulting data can be provided as input to the regression model or regressor as (Y=[X, [Image]]).

Feature Identification from Satellite Images

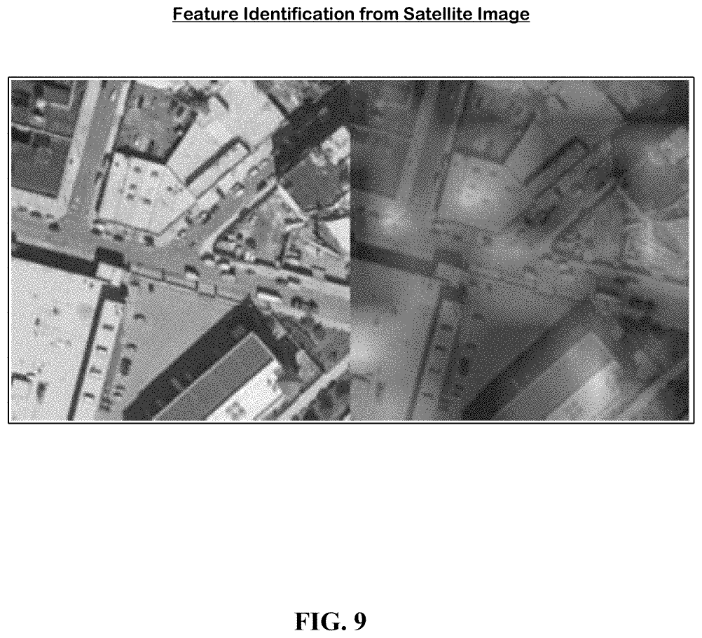

FIG. 9 is an example satellite image of a neighborhood (left). Image on the right is an activation map from convolutional layer of the artificial intelligence-implemented feature extractor such as AlexNet. The convolutional neural network (CNN) understands image by interpreting the output from filters learned during the training process. The activation maps may not align exactly with the original image owing to padding of output within the CNN. The technology disclosed trains the deep learning pipelines to determine what features of the environment are being focused on by the artificial intelligence-implemented feature extractor. The image in FIG. 9 shows that the model is identifying a number of large, dense building in the city block and correlating these features to the health indicator. A community with a higher density of buildings and highways can have a higher health risk for asthma. A community with many walking paths would have greater access to physical activity and lower risk for heart disease.

### Deep Learning Pipeline

In the following sections, we present details of the artificial intelligence models used in the deep learning pipeline. Two main tasks performed by models in the deep learning pipeline include feature extraction and prediction of prevalences of diseases and risk factors in geographical regions. Details of the models deployed for these two tasks are presented below.

### Feature Extraction Using Artificial Intelligence-Implemented Method

We present examples of artificial intelligence-implemented features extractors used in the deep learning pipeline.

AlexNet Feature Extractor

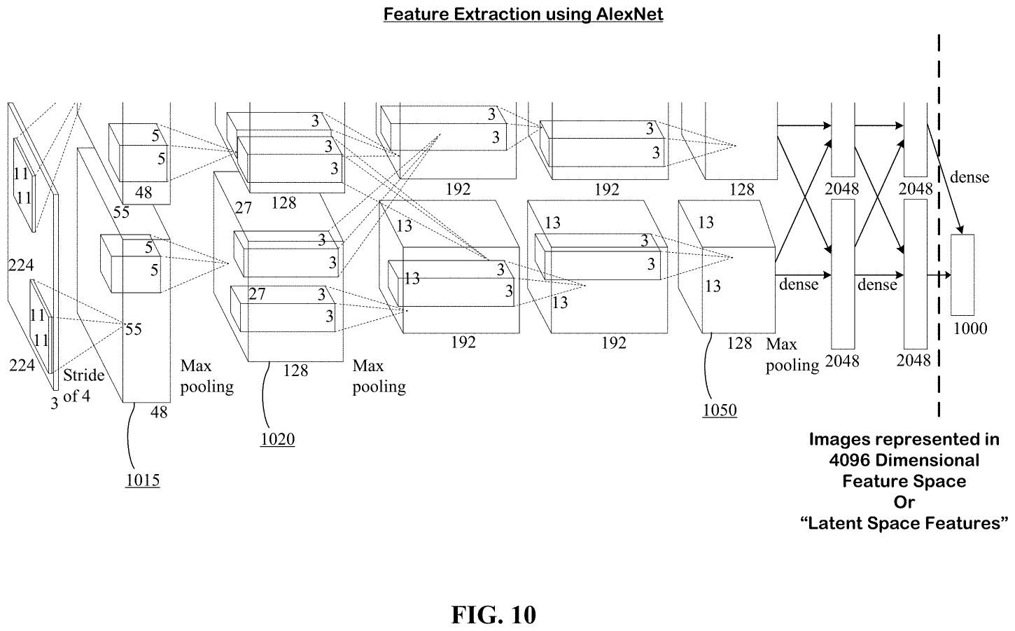

FIG. 10 is an example image feature extractor configured to process a plurality of satellite images for a particular census tract. The image feature generator can generate respective latent feature vectors for respective satellite images in the plurality of satellite images. The latent feature vectors can encode built environment of the particular census tract. The example image features extractor shown in FIG. 10 is a convolutional neural network (CNN) based model known as AlexNet (Krizhevsky et al., 2012, “Imagenet classification with deep convolutional neural networks”, published in Advances in Neural Information Processing Systems).

The system uses a pretrained AlexNet model. During pretraining, the AlexNet (CNN) model parameters are pretrained on ImageNet dataset (Deng et al. 2009, “ImageNet: A large-scale hierarchical image database”, published in proceedings of IEEE Conference on Computer Vision and Pattern Recognition, pp. 248-255) which contains around 14 million images. In many real-world applications, an entire convolutional neural network (CNN) is not trained from scratch with random initialization. This is because in most cases training datasets can be small. It is common to pretrain a CNN on a large dataset such as ImageNet which contains around 14 million images with 1000 categories (available at image-net.org) and then use the pretrained CNN as an initialization or a fixed feature extractor for the task of interest. This process is known as transfer learning to migrate the knowledge learned from the source dataset to a target dataset.

Transfer learning involves fine-tuning the pre-trained CNN for a new task or using the pretrained CNN for feature extraction combined with linear classification or regression. We process the satellite images of built environment per census tract through the pretrained AlexNet to extract features in a 4096-dimensional feature space. These features are referred to as “latent space features”. FIG. 10 illustrates output from the feature extractor taken before the last fully connected layer.

As shown in FIG. 10, the AlexNet model contains eight layers with weights; the first five are convolutional and the remaining three are fully-connected (FC). The output of the last fully-connected layer is fed to a 1000-way softmax which produces a distribution over the 1000 class labels. Our network maximizes the multinomial logistic regression objective, which is equivalent to maximizing the average across training cases of the log-probability of the correct label under the prediction distribution.

We extract images from the last layer before the feeding to softmax function. The extracted features are referred to as latent space features in a 4096-dimensional feature space. These vectors represent the elements of the environment that are detectable from the satellite images. Such features include buildings, roads, highways, trees, parks, sidewalks, walking paths, and farmland. These features are indicative of the exposures in a community that contribute to the community's health and disease risk. For example, a community with a higher density of buildings and highways would have a higher health risk for asthma. Similarly, a community with many walking paths would have greater access to physical activity, and lower risk for heart disease.

Referring to FIG. 10, the first convolutional layer filters the 224×224×3 input image with 96 kernels of size 11×11×3 with a stride of 4 pixels (this is the distance between the receptive field centers of neighboring neurons in a kernel map). The second convolutional layer takes as input the (response-normalized and pooled) output of the first convolutional layer and filters it with 256 kernels of size 5×5×48. The third, fourth, and fifth convolutional layers are connected to one another without any intervening pooling or normalization layers. The third convolutional layer has 384 kernels of size 3×3×256 connected to the (normalized, pooled) outputs of the second convolutional layer. The fourth convolutional layer has 384 kernels of size 3×3×192, and the fifth convolutional layer has 256 kernels of size 3×3×192. The fully-connected layers have 4096 neurons each.

The kernels of the second, fourth, and fifth convolutional layers are connected only to those kernel maps in the previous layer which may reside on the same GPU (see FIG. 10). The kernels of the third convolutional layer are connected to all kernel maps in the second layer. The neurons in the fully-connected (FC) layers are connected to all neurons in the previous layer. Response-normalization layers follow the first and second convolutional layers. Max-pooling layers follow both response-normalization layers as well as the fifth convolutional layer. The ReLU non-linearity is applied to the output of every convolutional and fully-connected layer.

The output of the first (1015), second (1020), and fifth (1050) convolutional layers are passed through pooling layers. The output of the convolution is also referred to as feature maps. This output is given as input to a max pool layer. The goal of a pooling layer is to reduce the dimensionality of feature maps. For this reason, it is also called “downsampling”. The factor to which the downsampling will be done is called “stride” or “downsampling factor”. The pooling stride is denoted by “s”. In one type of pooling, called “max-pool”, the maximum value is selected for each stride. For example, consider max-pooling with s=2 is applied to a 12-dimensional vector x=[1, 10, 8, 2, 3, 6, 7, 0, 5, 4, 9, 2]. Max-pooling vector x with stride s=2 means we select the maximum value out of every two values starting from the index 0, resulting in the vector [10, 8, 6, 7, 5, 9]. Therefore, max-pooling vector x with stride s=2 results in a 6-dimensional vector. Max pool layer reduces dimensionality of output from convolution layers.

ResNet Feature Extractor

The system can apply other artificial intelligence-implemented feature extractors such as residual convolutional neural network (ResNet) to extract features and generate latent space features. The ResNet architecture (He et al. CVPR 2016 available at arxiv.org/abs/1512.03385) was designed to avoid many issues with very deep neural networks. Most predominately, the use of residual connections helps to overcome the vanishing gradient problem. We used pretrained ResNet-152 architecture and a smaller version pretrained ResNet-50 architecture. The ResNet-50 model consists of 5 stages each with a convolution and Identity block. Each convolution block has 3 convolution layers and each identity block also has 3 convolution layers. The output from the ResNet is similar to the output from AlexNet and is given as input to the regressor. The latent space features from ResNet-152 can be given as input to Gradient Boosted Decision Trees (GDBT) or Extreme Gradient Boosting (XGBoost) regressor and in case of ResNet-50 the output can be given to a multilayer perceptron (MLP) regressor. We present further details of the deep learning pipeline in the following sections.

### Regression Models (Regressors)

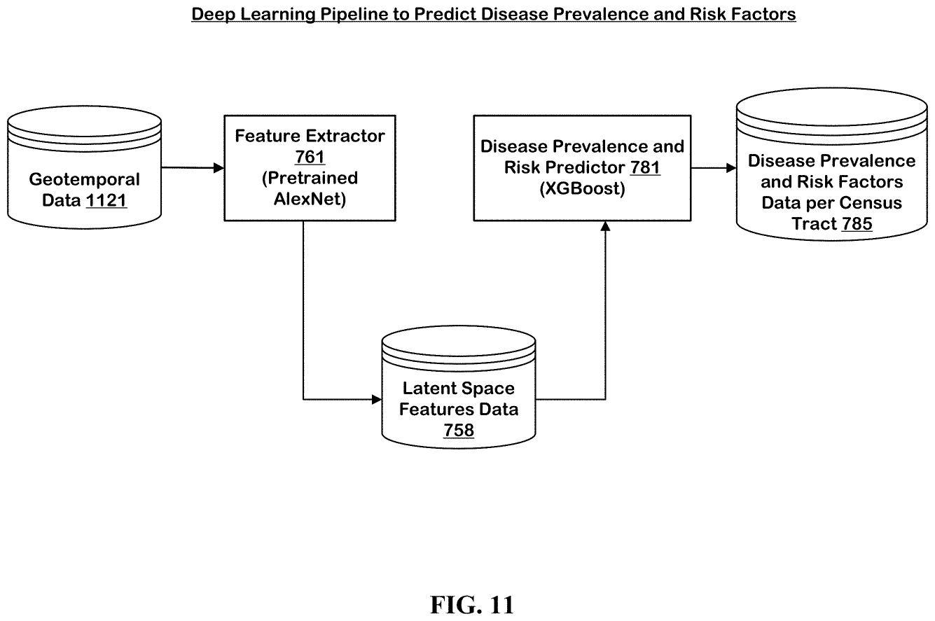

FIG. 11 presents a high-level architecture of the deep learning pipeline. The deep learning pipeline can perform artificial intelligence-implemented method of predicting comorbidity trajectories of disease categories on a census tract-basis. The deep learning pipeline can process a plurality of satellite images for a particular census tract and generate respective latent feature vectors for respective satellite images in the plurality of satellite images. The latent feature vectors encode built environment of the particular census tract. In this first step of processing, the geotemporal data 1121 is provided as input to feature extractor 761. In one implementation, the geotemporal data can include satellite images of built environment.

In another implementation, the satellite image data can be stored and accessed separately from the geotemporal data. The image data can be encoded with temporal information such as timestamps. The geotemporal data includes time series metrics for a plurality of environmental conditions over a time period. Environmental conditions can include pollution related data collected from sensors per unit of time such as hourly, daily, weekly, or monthly, etc. The geotemporal data includes time series metrics for a plurality of climate conditions over a time period. Climate conditions can indicate weather such cold, warm, etc. The climate related data can be collected from sensors over a period of time such as hourly, daily, weekly, or monthly, etc. The geotemporal data includes time series metrics for changes to a plurality of sociodemographic variables over a time period. For example, it can indicate changes in median income of population in a geographic area such as a census tract on a per yearly. The frequency of data collection can change without impacting the processing performed by the deep learning pipeline.

Latent space features in a 4096-dimensional image space are provided as input to regressors labeled as disease prevalence and risk predictor 781. Examples of regressors include extreme gradient boosting (XGBoost) or multilayer perceptrons (MLPs). Other types of regressor can be used such as gradient boosted decision trees (GDBT), random forest, etc.

XGBoost Regressor

Boosting and Bagging form the basis of several ensemble machine learning models. For example, random forest is an ensemble machine learning technique based on bagging. In bagging-based techniques, during training, subsamples of records are used to train different models such as decision trees in random forest. In addition, feature subsampling can also be used. The idea is that different models will be trained on different types of features and therefore, overall the model will perform well in production. The output of random forest is based on the output of individual models such as decision trees. The output from individual models is combined to produce the output from the random forest model.

The technology disclosed can use extreme gradient boosting or XGBoost regressor which is also an ensemble learning model. The boosting techniques are ensemble techniques that train machine learning models (such as decision trees) in sequential manner in such a way that each step of tree boosting improves the model performance. During training more weight is assigned to examples with incorrect prediction so that they have more chance of getting selected for the next model in the sequence. In addition, shrinkage and feature subsampling are used to reduce overfitting.

The latent space feature representation is regressed against the disease prevalence and risk features from CDC 500 Cities Project and the demographic factors from the American Community Survey using gradient boosted decision trees. We split the 80% of the data into training and the remaining 20% to testing. In one implementation, during training, we use a maximum tree depth of 5, a subsample of 80% of the features per tree, a learning rate (i.e., feature weight shrinkage for each boosting step) of 0.1, and used 3-fold cross-validation to determine the optimal number of boosted trees. Training was completed on a NVIDIA Tesla T4 GPU using Python 3.7.7 and the XGBoost package.

Shrinkage technique is used to prevent overfitting. It scales newly added weights by a factor (also referred to as learning rate) after each step of tree boosting. Similar to a learning rate in stochastic optimization, shrinkage reduces the influence of each individual tree and leaves space for future trees to improve the mode. Column subsampling (or feature subsampling) can also be used to prevent overfitting. In production, each decision tree produces a prediction. The final prediction for a given example is the sum of predictions from each tree (Chen et al. 2016, XGBoost: A Scalable Tree Boosting System).

The output from the regressors is a score for disease prevalence and risk factors. In one implementation, the regressors predict the prevalence values ranging from 0 to 1 for various diseases and risk factors from CDC 500 Cities data. The examples of diseases include Arthritis, Current Asthma, High Blood Pressure, Cancer (except skin), High Cholesterol, Kidney Disease, COPD, Heart Disease, Diabetes, Mental Health, Physical Health, Teeth Loss, Stroke, BP Medication, Obesity. Other examples of disease can be predicted by the technology disclosed. The examples of risk factors for which prevalence values can be predicted include Health Insurance, Annual Checkup, Dental Visit, Cholesterol Screening, Mammography, Pap Smear Test, Colorectal Cancer Screening, Preventive services (M), Preventive services (W), Binge Drinking, Smoker, Physical Inactivity, Sleep <7 hours. Other risk factors can be included for analysis.

Multilayer Perceptron (MLP) Regressor

Multilayer Perceptron (MLP) is a feed-forward neural network, the output layer and can have a single unit in case of regression. The system includes logic to perform regressing geotemporal data for the particular census tract and the respective weighted average latent feature vectors against the disease categories and the risk factors and generating the prevalence scores. In one implementation, we perform regression of the latent space feature representation against the disease prevalence and risk features from CDC 500 Cities Project using a Multilayer Perceptron (MLP). The MLP is sequential with three hidden layers. The first hidden layer has 1,024 nodes, the second has 512 nodes, and the third has 512 nodes. All layers have a ReLU activation function and a dropout layer with 10% probability. We split the data into 60% training, 20% validation, and 20% testing. The model is trained for the optimal number of epochs as determined by the validation set. We use the Adam optimizer and a learning rate of 0.0001 with 0.01 weight decay.

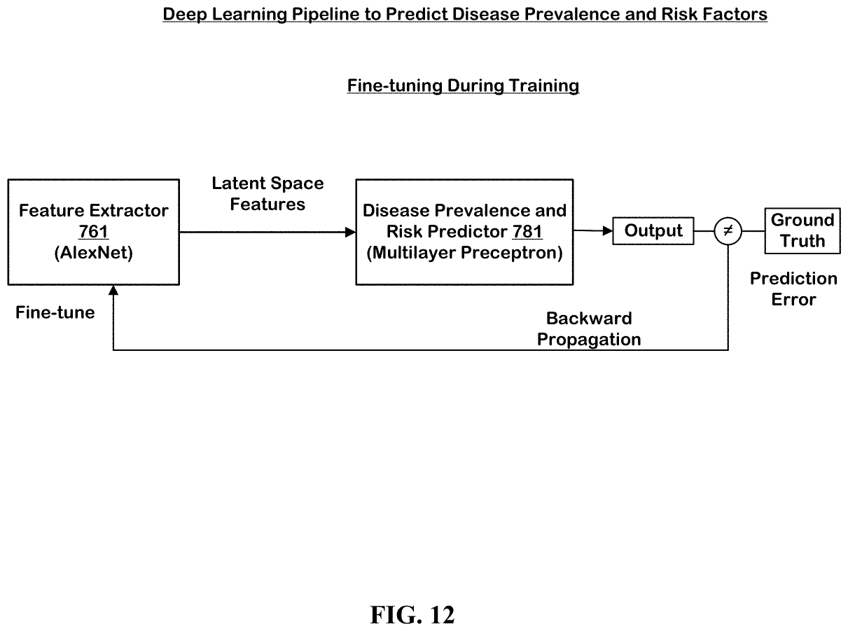

FIG. 12 presents fine-tuning of the feature extractor during training. In one implementation, fine-tuning of the mean latent space feature vector that is performed. The regression is performed using an MLP as described above. However, when training the MLP, the loss from prediction is backpropagated through the mean function and used to fine-tune (i.e., adjust the weights slightly) the AlexNet feature extractor. As a result, the latent space feature vector is no longer just the pretrained representation but rather is calculated as a function of the outcome variable. In this implementation, the MLP is sequential with two hidden layers. The first hidden layer has 512 nodes and the second has 256 nodes. All layers have a ReLU activation function and a dropout layer with 10% probability. We split the data into 60% training, 20% validation, and 20% testing. We use the Adam optimizer and a learning rate of 0.001 with 0.01 weight decay. We unfreeze the last convolutional layer from the pretrained AlexNet base to fine-tune.

Weighted Average Across Feature Vectors

The system includes logic to determine respective weighted average latent feature vectors for the respective latent feature vectors. The system can then regress the respective weighted average latent feature vectors against a plurality of disease categories and a plurality of risk factors and generate prevalence scores for disease categories in the plurality of disease categories and for risk factors in the plurality of risk factors. In this implementation, the system does not fine-tune the feature extractor. Instead, the system attempts to understand the importance of all the features in the latent space feature vector for each image and use this newfound knowledge in a learned weighting scheme rather than simply taking the mean over all the extracted feature vectors. Feature vectors are extracted from a pretrained AlexNet and are of size 4096 dimensions. The architecture is the same as that of the Multilayer Perceptron (MLP) described above, but additionally contains a 4096-dimension learned parameter vector that is dot product-ed with each image feature vector to produce an unnormalized image weight. The unnormalized image weights are then passed through a softmax function to get a normalized image weight over all images in a census tract. These normalized weights are used to take a weighted average of all the image feature vectors in a census tract.

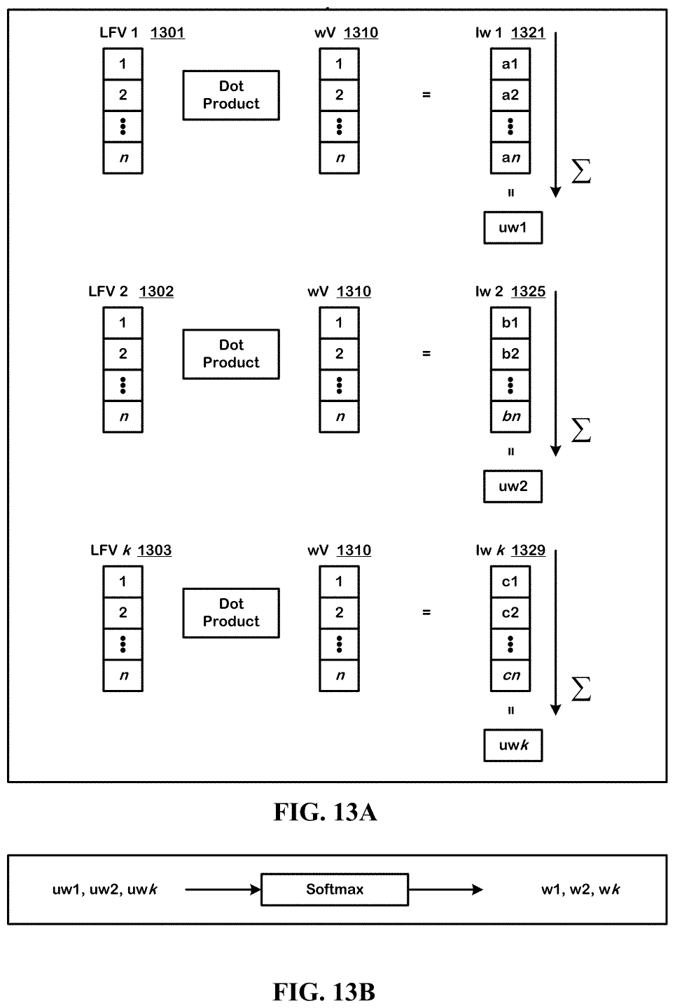

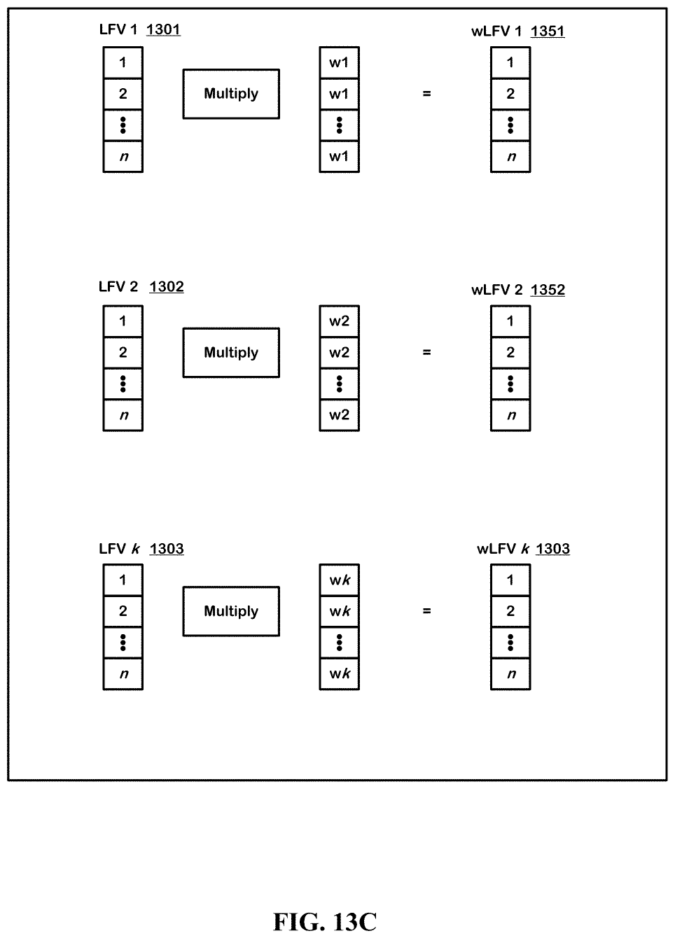

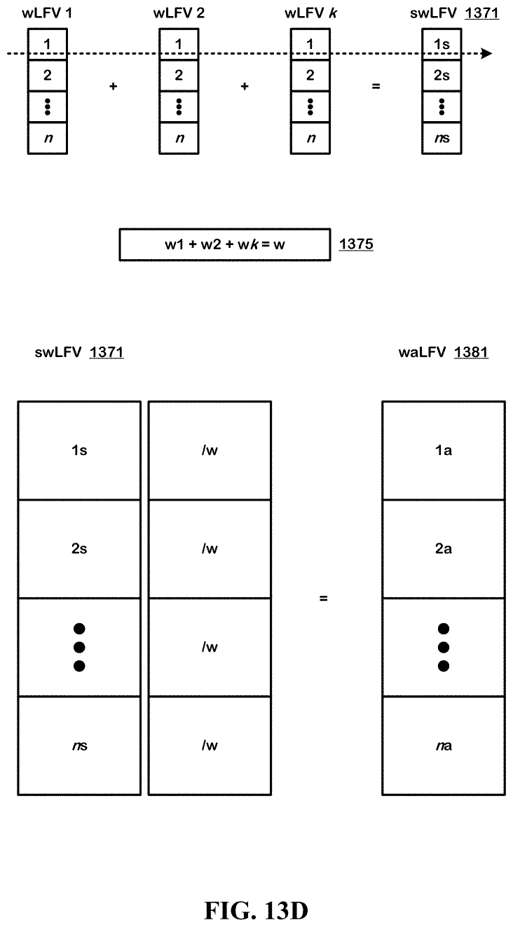

FIGS. 13A to 13D present a step wise process to calculate the weighted average latent feature vectors for respective latent space features. The process starts in FIG. 13A in which “k” laten space feature vectors or latent feature vectors for an image are shown in a vertical arrangement on the left side. The latent space feature vectors are output from the feature extractor such as AlexNet, ResNet, etc. The latent feature vectors are labeled as LFV 1 (1301), LFV 2 (1302), to LFV k (1303) where as “k” represents the number of images for a census tract. The value of k can be up to 250,000 or more. Each feature vector comprises of “n” values as shown in LFV 1, LFV 2, LFV k, where as “n” is the number of dimensions in the feature space. For example, in one implementation, the feature extractor to generate latent features in a 4096-dimensional image space, therefore, the value of “n” is 4096.

Each feature vector LFV 1, LFV 2, and LFV n are dot producted with a weighting vector wV (1310) as shown in FIG. 13A. This results in intermediate weights Iw 1 (1321), Iw 2 (1325), and Iw k (1329), for respective latent feature vectors. For example, when LFV 1 is dot producted with weighting vector (wV) the resulting output is intermediate weights Iw 1 (1321). An unnormalized weight (uw) is calculated for each intermediate vector by summing all weights in the respective intermediate weights. The unnormalized weights for respective for respective latent feature vectors are shown as uw1, uw2, uwk. The unnormalized weights for all latent feature vectors are passed through a softmax function to obtain normalized image weights labeled as w1, w2, wk, respectively for all latent feature vectors. A softmax is an exponential convex combinator configured to determine respective weighted average latent feature vectors for the respective latent feature vectors. In one implementation, the exponential convex combinator can use a weighting vector learned during training to calculate respective weights for the respective latent feature vectors, and determines the respective weighted average latent feature vectors by applying the respective weights to the respective latent feature vectors. Details of the softmax function are presented in the following section.

The latent feature vectors LFV 1, LFV 2, LFV k are multiplied with respective normalized weights w1, w2, wk as shown in FIG. 13C. This results in respective weighted latent feature vectors wLFV 1 (1351), wLFV 2 (1352), wLFV 3 (1353). A summation of weighted latent feature vectors is performed as shown in the top part of FIG. 13D. The results in a summed latent feature vector or swLFV 1371. As shown by the arrow, each element of the vection swLFV is a summation of respective elements in all weighted latent feature vectors for images for the census tract. Finally, a weighted average is calculated by dividing the elements of the swLFV by sum of normalized weights w1, w2, wk which is labeled as “w” (1375). This results in a weighted average latent feature vector or waLFV (1381). The system then uses the waLFV or weighted average latent feature vectors for when regressing against a plurality of disease categories and risk factors.

### Softmax Function

Softmax function is a preferred function for multi-class classification. The softmax function calculates the probabilities of each target class over all possible target classes. The output range of the softmax function is between zero and one and the sum of all the probabilities is equal to one. The softmax function computes the exponential of the given input value and the sum of exponential values of all the input values. The ratio of the exponential of the input value and the sum of exponential values is the output of the softmax function, referred to herein as “exponential normalization.”

Formally, training a so-called softmax classifier is regression to a class probability, rather than a true classifier as it does not return the class but rather a confidence prediction of each class's likelihood. The softmax function takes a class of values and converts them to probabilities that sum to one. The softmax function squashes a n-dimensional vector of arbitrary real values to n-dimensional vector of real values within the range zero to one. Thus, using the softmax function ensures that the output is a valid, exponentially normalized probability mass function (nonnegative and summing to one).

Intuitively, the softmax function is a “soft” version of the maximum function. The term “soft” derives from the fact that the softmax function is continuous and differentiable. Instead of selecting one maximal element, it breaks the vector into parts of a whole with the maximal input element getting a proportionally larger value, and the other getting a less proportion of the value. The property of outputting a probability distribution makes the softmax function suitable for probabilistic interpretation in classification tasks.

Let us consider z as a vector of inputs to the softmax layer. The softmax layer units are the number of nodes in the softmax layer and therefore, the length of the z vector is the number of units in the softmax layer (if we have ten output units, then there are ten z elements).

For an n-dimensional vector Z=[z₁, z₂, . . . zₙ] the softmax function uses exponential normalization (exp) to produce another n-dimensional vector p(Z) with normalized values in the range [0, 1] and that add to unity:

$$Z = [ \begin{matrix}{z}_{1} \\ {z}_{2} \\ ⋮ \\ {z}_{n}\end{matrix} ] and , p ( Z ) \to [ \begin{matrix}{p}_{1} \\ {p}_{2} \\ ⋮ \\ {p}_{n}\end{matrix} ]$$

$${p}_{j} = \frac{{exp}^{{z}_{j}}}{{{\sum }_{k = 1}}^{n} {exp}^{{z}_{k}}} ∀ j ∈ 1 , 2 , … , n$$

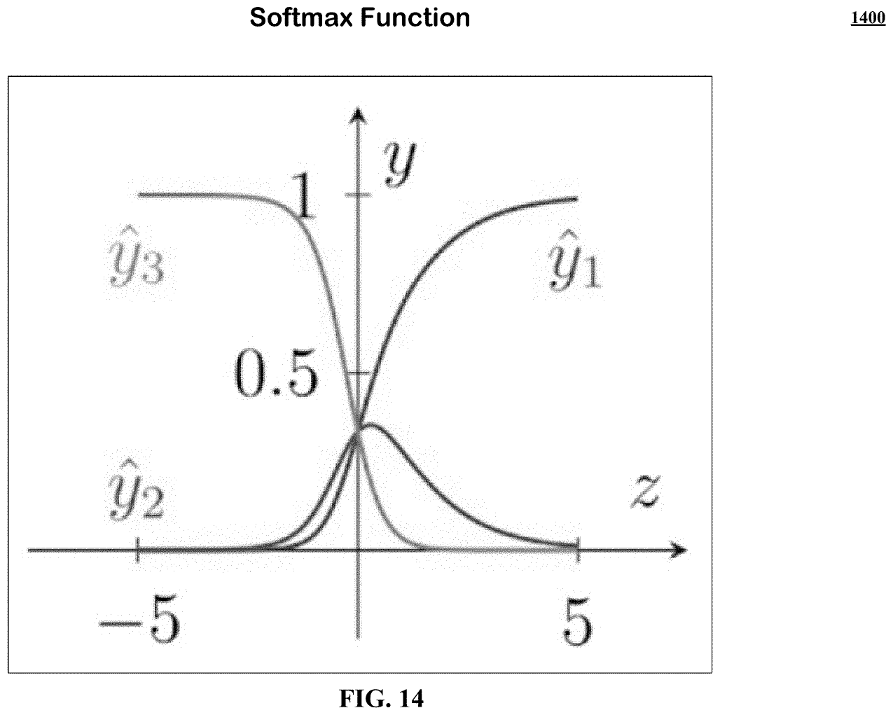

An example softmax function 1400 is shown in FIG. 14. Softmax function 1400 is applied to three classes as z [symbol] softmax

$$( [ z ; \frac{z}{10} ; - 2 z ] ) .$$

Note that the three outputs always sum to one. They thus define a discrete probability mass function.

### Prevalence Scores for Diseases and Risk Factors

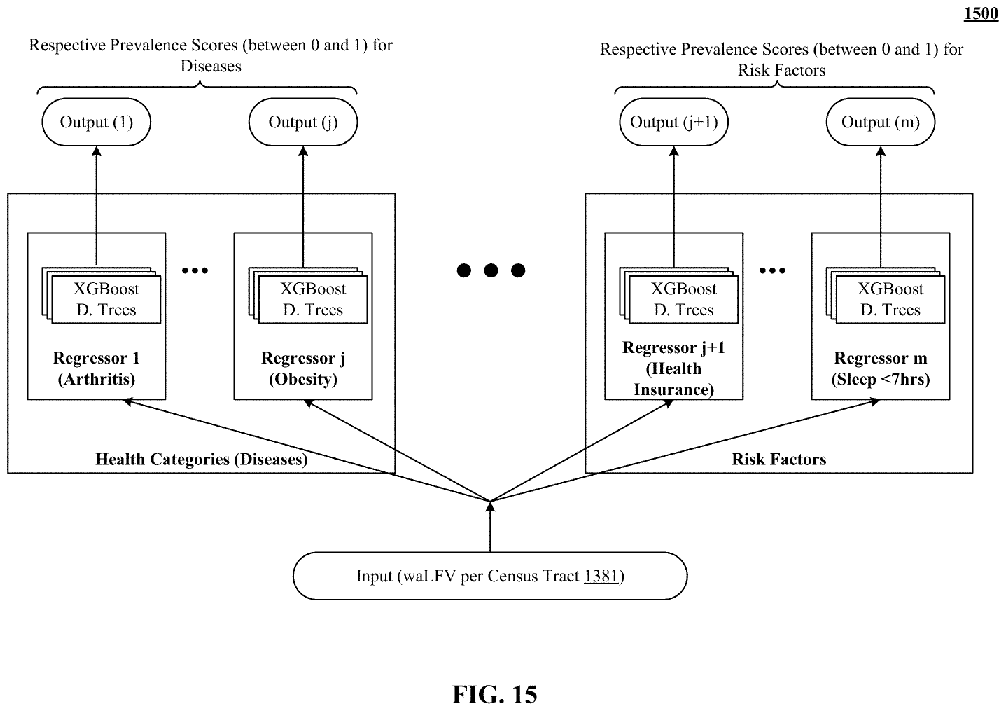

FIG. 15 presents an illustration 1500 of generating prevalence scores for diseases and risk factors using an example regressor (XGBoost). As explained above, other regressors such as MLP, random forest, etc. can also be used. The technology disclosed includes regression logic configured to regress the respective weighted average latent feature vectors against a plurality of disease categories and a plurality of risk factors. The regression logic can generate prevalence scores for disease categories in the plurality of disease categories and for risk factors in the plurality of risk factors. The regression logic can comprise respective regressors corresponding to respective disease categories and to respective risk factors. In FIG. 15, weighted average latent feature vector (waLFV 1381) per census tract is provided as input to “m” regressors.

Each regressor is trained to predict prevalence of a particular disease or risk factor. For example, regressor 1 is trained to predict prevalence score (between 0 and 1) for Arthritis. Regressors 1 to j can predict prevalence scores for respective diseases. In one implementation, there are 15 regressors which can respectively predict prevalence scores for 15 diseases: Arthritis, Current Asthma, High Blood Pressure, Cancer (except skin), High Cholesterol, Kidney Disease, COPD, Heart Disease, Diabetes, Mental Health, Physical Health, Teeth Loss, Stroke, BP Medication, Obesity. Similarly, regressors j+1 to m can predict prevalence scores for respective risk factors. In one implementation, there are 12 regressors which can respectively predict prevalence scores for 12 risk factors: Health Insurance, Annual Checkup, Dental Visit, Cholesterol Screening, Mammography, Pap Smear Test, Colorectal Cancer Screening, Preventive services (M), Preventive services (W), Binge Drinking, Smoker, Physical Inactivity, Sleep <7 hours.

The technology disclosed can performed other analysis using the deep learning pipeline. In one implementation, the system includes logic to regress the respective weighted average latent feature vectors against the disease categories, the risk factors, and the plurality of sociodemographic variables. The system generates prevalence scores for the disease categories and for risk factors across sociodemographic variables in the plurality of sociodemographic variables. In this analysis, the system can predict prevalence of diseases and risk factors in different segments or groups of population in a census tract. For example, the prevalence of arthritis in males and females or the prevalence of diabetes in individuals of different races. Thus, the system can correlate the disease categories with each other and with the risk factors based on the prevalence scores and determine the comorbidity trajectories of the disease categories in the particular census tract across the sociodemographic variables.

The system can use a bootstrap-based approach or standard normal (en.wikipedia.org/wiki/Prediction_interval) to correlate predictions of regressors or predictors. The system can estimate a standard error of the predictions and for each census tract, estimate a prediction interval. This interval can define the range of the prediction for a new census tract with similar attributes of the built environment.

In another implementation, the system includes logic to regress the geotemporal data and the respective weighted average latent feature vectors against the disease categories, the risk factors, and the sociodemographic variables and generating the prevalence scores. As described above geotemporal data can include time series metrics for environmental conditions (e.g., air pollution), climate conditions (e.g., weather), or time series metrics for a plurality of sociodemographic variables over a time period. For example, it can indicate changes in median income of population in a geographic area such as a census tract on a per yearly. In this case the system can generate outputs for sociodemographic variables when such data is not available for a given geographical location such as a census tract. This output can be compared with sociodemographic variables of other census tracts for further analysis.

In another implementation, the system can include logic to regress the sociodemographic variables and the respective weighted average latent feature vectors against the disease categories and the risk factors and generating the prevalence scores. In this implementation, the satellite image features as represented by their respective weighted average latent feature vectors and the social determinants of health features from the American Community Survey are regressed against the CDC 500.

### Particular Implementations

We describe implementations of a system for predicting comorbidity trajectories of disease categories on a census tract-basis.

The technology disclosed can be practiced as a system, method, or article of manufacture. One or more features of an implementation can be combined with the base implementation. Implementations that are not mutually exclusive are taught to be combinable. One or more features of an implementation can be combined with other implementations. This disclosure periodically reminds the user of these options. Omission from some implementations of recitations that repeat these options should not be taken as limiting the combinations taught in the preceding sections—these recitations are hereby incorporated forward by reference into each of the following implementations.

A method implementation of the technology disclosed includes an artificial intelligence-implemented method of predicting comorbidity trajectories of disease categories on a census tract-basis. The method includes processing a plurality of satellite images for a particular census tract and generating respective latent feature vectors for respective satellite images in the plurality of satellite images. The latent feature vectors can encode built environment of the particular census tract. The method includes determining respective weighted average latent feature vectors for the respective latent feature vectors. The method includes regressing the respective weighted average latent feature vectors against a plurality of disease categories and a plurality of risk factors. The method includes generating prevalence scores for disease categories in the plurality of disease categories and for risk factors in the plurality of risk factors. The method includes correlating the disease categories with each other and with the risk factors based on the prevalence scores and determining the comorbidity trajectories of the disease categories in the particular census tract.

This method implementation and other methods disclosed optionally include one or more of the following features. This method can also include features described in connection with systems disclosed. In the interest of conciseness, alternative combinations of method features are not individually enumerated. Features applicable to methods, systems, and articles of manufacture are not repeated for each statutory class set of base features. The reader will understand how features identified in this section can readily be combined with base features in other statutory classes.

In one implementation, the artificial intelligence-implemented method described above further includes regressing geotemporal data for the particular census tract and for a given time period and the respective weighted average latent feature vectors against the disease categories and the risk factors and generating the prevalence scores for the given time period.

The geotemporal data includes time series metrics for a plurality of environmental conditions over the given time period.

The geotemporal data includes time series metrics for a plurality of climate conditions over the given time period.

The geotemporal data includes time series metrics for changes to a plurality of sociodemographic variables over the given time period. In such an implementation, the method further includes regressing the respective weighted average latent feature vectors against the disease categories, the risk factors, and the plurality of sociodemographic variables. The method includes generating the prevalence scores for the disease categories and for risk factors across sociodemographic variables in the plurality of sociodemographic variables. The method includes correlating the disease categories with each other and with the risk factors based on the prevalence scores and determining the comorbidity trajectories of the disease categories in the particular census tract across the sociodemographic variables.

The method further includes regressing the geotemporal data and the respective weighted average latent feature vectors against the disease categories, the risk factors, and the sociodemographic variables and generating the prevalence scores. In such an implementation, values for the geotemporal data are appended to the respective weighted average latent feature vectors to produce a combined input, and the regressing is executed on the combined input.

The method further includes regressing the sociodemographic variables and the respective weighted average latent feature vectors against the disease categories and the risk factors and generating the prevalence scores.

The values for the sociodemographic variables are appended to the respective weighted average latent feature vectors to produce a combined input, and the regressing is executed on the combined input. The plurality of satellite images is captured for the given time period.

The method includes regressing the respective weighted average latent feature vectors against the geotemporal data and generating predicted scores for the time series metrics for the plurality of environmental conditions, the plurality of climate conditions, and the plurality of sociodemographic variables for the particular census tract and for the given time period.

The sociodemographic variables are measured for the given time period.

The method includes regressing the sociodemographic variables and the respective weighted average latent feature vectors against the geotemporal data and generating the predicted scores.

The geotemporal data can include time series metrics for a plurality of environmental conditions over a time period. The geotemporal data can include time series metrics for a plurality of climate conditions over a time period. The geotemporal data can include time series metrics for changes to a plurality of sociodemographic variables over a time period.

Other implementations may include a non-transitory computer readable storage medium storing instructions executable by a processor to perform a method as described above. Yet another implementation may include a system including memory and one or more processors operable to execute instructions, stored in the memory, to perform a method as described above.

Computer readable media (CRM) implementations of the technology disclosed include a non-transitory computer readable storage medium impressed with computer program instructions, when executed on a processor, implement the methods described above.

Each of the features discussed in this particular implementation section for the method implementation apply equally to the CRM implementation. As indicated above, all the system features are not repeated here and should be considered repeated by reference.

A system implementation of the technology disclosed includes one or more processors coupled to memory. The memory is loaded with computer instructions to predicting comorbidity trajectories of disease categories on a census tract-basis. The artificial intelligence-implemented system includes an image feature extractor configured to process a plurality of satellite images for a particular census tract and generate respective latent feature vectors for respective satellite images in the plurality of satellite images. The latent feature vectors encode built environment of the particular census tract. The system includes an exponential convex combinator configured to determine respective weighted average latent feature vectors for the respective latent feature vectors. The system includes regression logic configured to regress the respective weighted average latent feature vectors against a plurality of disease categories and a plurality of risk factors. The system includes logic to generate prevalence scores for disease categories in the plurality of disease categories and for risk factors in the plurality of risk factors. The regression logic comprises respective regressors corresponding to respective disease categories and to respective risk factors. The system includes a correlator configured to correlate the disease categories with each other and with the risk factors based on the prevalence scores and determine the comorbidity trajectories of the disease categories in the particular census tract.

This system implementation optionally include one or more of the following features. This system can also include features described in connection with methods disclosed above. In the interest of conciseness, alternative combinations of method features are not individually enumerated. Features applicable to methods, systems, and articles of manufacture are not repeated for each statutory class set of base features. The reader will understand how features identified in this section can readily be combined with base features in other statutory classes.

The regressors can be gradient boosted decision trees (GBDT), or eXtreme gradient boosting (XGBoost), or random forest trees or multilayer perceptrons (MLPs). The image feature extractor can be a convolution neural network such as AlexNet. The image feature extractor can be a residual convolution neural network such as ResNet.

The exponential convex combinator can uses a weighting vector learned during training to calculate respective weights for the respective latent feature vectors. The e exponential convex combinator can determine the respective weighted average latent feature vectors by applying the respective weights to the respective latent feature vectors.

The correlator is further configured to identify those disease categories and risk factors with prevalence scores within a threshold range, and to infer shared dependencies between the disease categories and the risk factors.

Other implementations may include a non-transitory computer readable storage medium storing instructions executable by a processor to perform functions of the system described above. Yet another implementation may include a method performing the functions of the system described above.

A computer readable storage medium (CRM) implementation of the technology disclosed includes a non-transitory computer readable storage medium impressed with computer program instructions to generate a multi-part place identifier with at least one part. The instructions when executed on a processor, implement the method described above.

Each of the features discussed in this particular implementation section for the method implementation apply equally to the CRM implementation. As indicated above, all the method features are not repeated here and should be considered repeated by reference.

### Particular Implementations—Federated Learning

The technology disclosed includes a system federated learning model for healthcare application in association with one or more geotemporal factors. The system includes a raw data collector, configured to collect geographical data associated with time identifier from one or more database. The system includes a data integrator, configured to integrate geographical data with corresponding time identifier. The system includes an image feature extractor, configured to extract geotemporal information and reduce dimensions of vectors. The system includes a data merger, configured to merge geotemporal information with geometric shape information. The system includes a cohort builder, configured to build cohort of data based on criteria of data inclusion from pool of data of data merger, one or more cloud, or one or more edge device. The system includes a distributed learning network, configured to learn from a plurality of tensors sent by the one or more edge device or one or more cloud server. The system includes a geotemporal machine learning model aggregator, configured to receive averaged value of the plurality of tensors to update the geotemporal machine learning model.

The technology disclosed implements a method with federated learning model for healthcare application in association with one or more geotemporal factor. The method includes collecting, geographical data associated with time identifier from one or more database. The method includes integrating, geographical data with corresponding time identifier. The method includes extracting, geotemporal information from geographical information with time identifier. The method includes reducing, dimensions of vectors of the geotemporal information. The method includes merging, geotemporal information with geometric shape information. The method includes building, cohort of data based on criteria of data inclusion from pool of data of merged data, one or more cloud, and one or more edge device. The method includes training, a geotemporal machine learning model in a distributed learning network by a plurality of tensors sent by the one or more edge device. The method includes updating, the geotemporal machine learning model by averaged value of the plurality of tensors.

The technology disclosed include a system to integrate geotemporal data. The system includes one or more database, having geographical data associated with time identifier, wherein the geographical data having geotemporal factors. The system includes a geotemporal data integrator, configured to integrate geographical data with corresponding time identifier. The system includes an image feature extractor, configured to extract geotemporal information and reduce dimensions of vectors. The system includes a data merger, configured to merge geotemporal information with geometric shape information. The data merger further includes an application programming interface configured to interact with external cloud or edge device.

The technology disclosed implements a method to integrate geotemporal data. The method includes receiving, geographical data associated with time identifier from one or more database, wherein the geographical data having geotemporal factors. The method includes integrating geotemporal data with corresponding time identifier. The method includes extracting image features with geotemporal information and reduced dimensions of vectors. The method includes merging geotemporal information with geometric shape information. The method includes interacting by an application programming interface with external one or more cloud or edge device.

### Computer System

FIG. 16 is a simplified block diagram of a computer system 1600 that can be used to implement the technology disclosed. Computer system typically includes at least one processor 1672 that communicates with a number of peripheral devices via bus subsystem 1655. These peripheral devices can include a storage subsystem 1610 including, for example, memory subsystem 1622 and a file storage subsystem 1636, user interface input devices 1638, user interface output devices 1676, and a network interface subsystem 1674. The input and output devices allow user interaction with computer system. Network interface subsystem provides an interface to outside networks, including an interface to corresponding interface devices in other computer systems.

User interface input devices 1638 can include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system.

User interface output devices 1676 can include a display subsystem, a printer, a fax machine, or non-visual displays such as audio output devices. The display subsystem can include a cathode ray tube (CRT), a flat-panel device such as a liquid crystal display (LCD), a projection device, or some other mechanism for creating a visible image. The display subsystem can also provide a non-visual display such as audio output devices. In general, use of the term “output device” is intended to include all possible types of devices and ways to output information from computer system to the user or to another machine or computer system.

Storage subsystem 1610 stores programming and data constructs that provide the functionality of some or all of the modules and methods described herein. These software modules are generally executed by processor alone or in combination with other processors.

Memory used in the storage subsystem can include a number of memories including a main random access memory (RAM) 1632 for storage of instructions and data during program execution and a read only memory (ROM) 1634 in which fixed instructions are stored. The file storage subsystem 1636 can provide persistent storage for program and data files, and can include a hard disk drive, a floppy disk drive along with associated removable media, a CD-ROM drive, an optical drive, or removable media cartridges. The modules implementing the functionality of certain implementations can be stored by file storage subsystem in the storage subsystem, or in other machines accessible by the processor.

Bus subsystem 1655 provides a mechanism for letting the various components and subsystems of computer system communicate with each other as intended. Although bus subsystem is shown schematically as a single bus, alternative implementations of the bus subsystem can use multiple busses.

Computer system itself can be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the description of computer system depicted in FIG. 16 is intended only as a specific example for purposes of illustrating the technology disclosed. Many other configurations of computer system are possible having more or less components than the computer system depicted in FIG. 16.

The computer system 1600 includes GPUs or FPGAs 1678. It can also include machine learning processors hosted by machine learning cloud platforms such as Google Cloud Platform, Xilinx, and Cirrascale. Examples of deep learning processors include Google's Tensor Processing Unit (TPU), rackmount solutions like GX4 Rackmount Series, GX8 Rackmount Series, NVIDIA DGX-1, Microsoft' Stratix V FPGA, Graphcore's Intelligent Processor Unit (IPU), Qualcomm's Zeroth platform with Snapdragon processors, NVIDIA's Volta, NVIDIA's DRIVE PX, NVIDIA's JETSON TX1/TX2 MODULE, Intel's Nirvana, Movidius VPU, Fujitsu DPI, ARM's DynamicIQ, IBM TrueNorth, and others.

We claim as follows:

1. An artificial intelligence-implemented method of predicting comorbidity trajectories of disease categories on a census tract-basis, including:
processing a plurality of satellite images for a particular census tract and generating respective latent feature vectors for respective satellite images in the plurality of satellite images, wherein the latent feature vectors encode built environment of the particular census tract;
determining respective weighted average latent feature vectors for the respective latent feature vectors;
regressing the respective weighted average latent feature vectors against a plurality of disease categories and a plurality of risk factors and generating prevalence scores for disease categories in the plurality of disease categories and for risk factors in the plurality of risk factors; and
correlating the disease categories with each other and with the risk factors based on the prevalence scores and determining the comorbidity trajectories of the disease categories in the particular census tract.

2. The artificial intelligence-implemented method of claim 1, further including regressing geotemporal data for the particular census tract and for a given time period and the respective weighted average latent feature vectors against the disease categories and the risk factors and generating the prevalence scores for the given time period.

3. The artificial intelligence-implemented method of claim 2, wherein the geotemporal data includes time series metrics for a plurality of environmental conditions over the given time period.

4. The artificial intelligence-implemented method of claim 2, wherein the geotemporal data includes time series metrics for a plurality of climate conditions over the given time period.

5. The artificial intelligence-implemented method of claim 2, wherein the geotemporal data includes time series metrics for changes to a plurality of sociodemographic variables over the given time period.

6. The artificial intelligence-implemented method of claim 5, further including:
regressing the respective weighted average latent feature vectors against the disease categories, the risk factors, and the plurality of sociodemographic variables and generating the prevalence scores for the disease categories and for risk factors across sociodemographic variables in the plurality of sociodemographic variables; and
correlating the disease categories with each other and with the risk factors based on the prevalence scores and determining the comorbidity trajectories of the disease categories in the particular census tract across the sociodemographic variables.

7. The artificial intelligence-implemented method of claim 6, further including regressing the geotemporal data and the respective weighted average latent feature vectors against the disease categories, the risk factors, and the sociodemographic variables and generating the prevalence scores.

8. The artificial intelligence-implemented method of claim 7, wherein values for the geotemporal data are appended to the respective weighted average latent feature vectors to produce a combined input, and the regressing is executed on the combined input.

9. The artificial intelligence-implemented method of claim 6, further including regressing the sociodemographic variables and the respective weighted average latent feature vectors against the disease categories and the risk factors and generating the prevalence scores.

10. The artificial intelligence-implemented method of claim 9, wherein values for the sociodemographic variables are appended to the respective weighted average latent feature vectors to produce a combined input, and the regressing is executed on the combined input.

11. The artificial intelligence-implemented method of claim 2, wherein the plurality of satellite images is captured for the given time period.

12. The artificial intelligence-implemented method of claim 11, further including regressing the respective weighted average latent feature vectors against the geotemporal data and generating predicted scores for the time series metrics for the plurality of environmental conditions, the plurality of climate conditions, and the plurality of sociodemographic variables for the particular census tract and for the given time period.

13. The artificial intelligence-implemented method of claim 2, wherein the sociodemographic variables are measured for the given time period.

14. The artificial intelligence-implemented method of claim 13, further including regressing the sociodemographic variables and the respective weighted average latent feature vectors against the geotemporal data and generating the predicted scores.

15. An artificial intelligence-based system for predicting comorbidity trajectories of disease categories on a census tract-basis, including:
an image feature extractor configured to process a plurality of satellite images for a particular census tract and generate respective latent feature vectors for respective satellite images in the plurality of satellite images, wherein the latent feature vectors encode built environment of the particular census tract;
an exponential convex combinator configured to determine respective weighted average latent feature vectors for the respective latent feature vectors;
regression logic configured to regress the respective weighted average latent feature vectors against a plurality of disease categories and a plurality of risk factors and generate prevalence scores for disease categories in the plurality of disease categories and for risk factors in the plurality of risk factors, wherein the regression logic comprises respective regressors corresponding to respective disease categories and to respective risk factors; and
a correlator configured to correlate the disease categories with each other and with the risk factors based on the prevalence scores and determine the comorbidity trajectories of the disease categories in the particular census tract.

16. The artificial intelligence-based system of claim 15, wherein the regressors are gradient boosted decision trees (GBDT).

17. The artificial intelligence-based system of claim 16, wherein the regressors are eXtreme gradient boosting (XGBoost).

18. The artificial intelligence-based system of claim 15, wherein the regressors are random forest trees.

19. The artificial intelligence-based system of claim 15, wherein the regressors are multilayer perceptrons (MLPs).

20. The artificial intelligence-based system of claim 15, wherein the image feature extractor is a convolution neural network.

21. The artificial intelligence-based system of claim 15, wherein the image feature extractor is a residual convolution neural network.

22. The artificial intelligence-based system of claim 15, wherein the exponential convex combinator uses a weighting vector learned during training to calculate respective weights for the respective latent feature vectors, and determines the respective weighted average latent feature vectors by applying the respective weights to the respective latent feature vectors.

23. The artificial intelligence-based system of claim 15, wherein the correlator is further configured to identify those disease categories and risk factors with prevalence scores within a threshold range, and to infer shared dependencies between the disease categories and the risk factors.

24. A non-transitory computer readable storage medium impressed with computer program instructions to predict comorbidity trajectories of disease categories on a census tract-basis, the instructions, when executed on a processor, implement a method comprising, including:
processing a plurality of satellite images for a particular census tract and generating respective latent feature vectors for respective satellite images in the plurality of satellite images, wherein the latent feature vectors encode built environment of the particular census tract;
determining respective weighted average latent feature vectors for the respective latent feature vectors;
regressing the respective weighted average latent feature vectors against a plurality of disease categories and a plurality of risk factors and generating prevalence scores for disease categories in the plurality of disease categories and for risk factors in the plurality of risk factors; and
correlating the disease categories with each other and with the risk factors based on the prevalence scores and determining the comorbidity trajectories of the disease categories in the particular census tract.

25. The non-transitory computer readable storage medium of claim 24, implementing the method further comprising, regressing geotemporal data for the particular census tract and for a given time period and the respective weighted average latent feature vectors against the disease categories and the risk factors and generating the prevalence scores for the given time period.
